(19) United States
(12) Patent Application Publication
(10) Pub. No.: US 2005/0194561 A1
(43) Pub. Date: Sep. 8, 2005

(54) Anionic-sweetener-based ionic liquids and methods of use thereof

(75) Inventor: James H. Davis JR., Mobile, AL (US)

Correspondence Address: FOLEY HOAG, LLP;PATENT GROUP, WORLD TRADE CENTER WEST, 155 SEAPORT BLVD, BOSTON, MA, 02110, US

(73) Assignee: University of South Alabama, Mobile, AL (US)

(21) Appl. No.: 11/046,561

(22) Filed: Jan. 26, 2005

Related U.S. Application Data
(60) Provisional application No. 60/539,870, filed on Jan. 26, 2004.

(51) Int. Cl. F25D 1/00; C09K 5/00; C07D 417/02

(52) U.S. Cl. 252/67; 544/333; 546/268.1

(57) ABSTRACT

One aspect of the present invention relates to ionic liquids comprising an anionic sweetener. Another aspect of the present invention relates to the use of an ionic liquid of the present invention comprising a pendant Bronsted-acidic group to catalyze a Bronsted-acid-catalyzed chemical reaction. A third aspect of the present invention relates to ionic liquids of the present invention comprising a pendant nucleophilic group, e.g., an amine. Still another aspect of the present invention relates to the use of an ionic liquid of the present invention comprising a pendant nucleophilic group to catalyze a nucleophile-assisted chemical reaction. A fifth aspect of the present invention relates to the use of an ionic liquid of the present invention comprising a pendant nucleophilic group to remove a gaseous impurity, e.g., carbon dioxide, from a gas, e.g., sour natural gas. A sixth aspect of the present invention relates to heat storage media comprising an ionic liquid of the present invention.

### RELATED APPLICATIONS

This application claims the benefit of priority to U.S. Provisional Patent Application Ser. No. 60/539,870, filed Jan. 26, 2004; the contents of which are incorporated by reference.

### BACKGROUND OF THE INVENTION

Ionic Liquids

Ionic liquids consist of ions. However, unlike conventional molten salts (for example, molten sodium chloride), ionic liquids often melt below 100° C. When an ionic liquid has a melting point below room temerature, it is said to be a room-temperature ionic liquid. Since their melting points are low, room-temperature ionic liquids can act as solvents in which reactions can be performed. Because an ionic liquid is made of ions rather than molecules, they often provide distinct selectivities and reactivities as compared to conventional organic solvents.

Room-temperature ionic liquids have been used as clean solvents and catalysts for green chemistry and as electrolytes for batteries, photochemistry and electrosynthesis. They have no significant vapor pressure and thus create no volatile organic contaminants. They also allow for easy separation of organic molecules by direct distillation without loss of the ionic liquid. Their liquid range can be as large as 300° C. allowing for large reaction kinetic control, which, coupled with their good solvent properties, allows small reactor volumes to be used. Salts based upon poor nucleophilic anions, such as [BF₄]⁻, [PF₆]⁻, [CF₃CO₂]⁻, and [CF₃SO₃]⁻, are water and air insensitive and possess remarkably high thermal stability. Many of these materials are based around an imidazolium cation, 1-alkyl-3-methylimidazolium. By changing the anion or the alkyl chain on the cation, a wide variation in properties, such as hydrophobicity, viscosity, density and solvation, can be obtained. For example, ionic liquids will dissolve a wide range of organic molecules to an appreciable extent, the solubility being influenced by the nature of the counter anion.

The unique physical properties of ionic liquids have been found to offer certain advantages in numerous applications. For example, U.S. Pat. No. 5,827,602 to Koch et al. discloses ionic liquids having improved properties for application in batteries, electrochemical capacitors, catalysis, chemical separations, and other uses. The ionic liquids described in Koch et al. are hydrophobic in nature, being poorly soluble in water, and contain only non-Lewis acid anions. When fluorinated, they were found to be particularly useful as hydraluic fluids and inert liquid diluents for highly reactive chemicals. In addition, ionic liquids have been discussed by Freemantle, M. Chem. Eng. News 1998, 76 [March 30], 32; Carmichael, H. Chem. Britain, 2000, [January], 36; Seddon, K. R. J. Chem. Tech. Biotechnol. 1997, 68, 351; Welton, T. Chem. Rev. 1999, 99, 2071; Bruce, D. W., Bowlas, C. J., Seddon, K. R. Chem. Comm. 1996, 1625; Merrigan, T. L., Bates, E. D., Dorman, S. C., Davis, J. H. Chem. Comm. 2000, 2051; Freemantle, M. Chem. Eng. News, 2000, 78 [May 15], 37. See also Holbrey, J. D.; Seddon, K. R. Clean Products and Processes 1999, 1, 223-236; and Dupont, J., Consorti, C. S. Spencer, J. J Braz. Chem. Soc. 2000, 11, 337-344.

Ionic liquids have been used as solvents for a broad spectrum of chemical processes. These ionic liquids, which in some cases serve as both catalyst and solvent, are attracting increasing interest from industry because they promise significant environmental benefits, e.g., because they are nonvolatile they do not emit vapors. Hence, for example, they have been used in butene dimerization processes. WO 95/21871, WO 95/21872 and WO 95/21806 relate to ionic liquids and their use to catalyse hydrocarbon conversion reactions, such as polymerisation and alkylation reactions. The ionic liquids described for this process were preferably 1-(C₁-C₄ alkyl)-3-(C₆-C₃₀ alkyl) imidazolium chlorides and especially 1-methyl-3-C₁₀ alkyl-imidazolium chloride, or 1-hydrocarbyl pyridinium halides, where the hydrocarbyl group is, for example, ethyl, butyl or other alkyl. PCT publication WO 01/25326 to Lamanna et al. discloses an antistatic composition comprising at least one ionic salt consisting of a nonpolymeric nitrogen onium cation and a weakly coordinating fluoroorganic anion, the conjugate acid of the anion being a superacid, in combination with thermoplastic polymer. The composition was found to exhibit good antistatic performance over a wide range of humidity levels.

However, it has been pointed out that touting the environmental benefits of IL chemistry is something that should be done with care. J. D. Holbrey, M. B. Turner and R. D. Rogers in Ionic Liquids as Green Solvents-Progress and Prospects; R. D. Rogers and K. R. Seddon, Eds.; ACS Symposium Series 856; American Chemical Society: Washington, D.C. 2003; 2. In a recent paper, a commentary has been offered on this situation as it pertains to fluorous anions, which are the most widely used anion type in IL formulations. R. P. Swatlowski, J. D. Holbrey and R. D. Rogers, Green Chem., 2003, 5, 361; L. Crowhurst, N. L. Lancaster, J. M. Perez-Arlandis, and T. Welton, advance copy of a manuscript dealing with ion effects in IL, submitted for publication in an ACS Symposium Series monograph on ionic liquids. While there are situations in which IL with fluorous anions will remain indispensible, there is much to be desired in identifying other (preferably innocuous) ions in formulating IL, especially for large-volume applications. J. H. Davis, Jr. and P. A. Fox, Chem. Commun., 2003, 1209; R. P. Swatlowski, J. D. Holbrey and R. D. Rogers, Green Chem., 2003, 5, 361; L. Crowhurst, N. L. Lancaster, J. M. Perez-Arlandis, and T. Welton, an advance copy of a manuscript dealing with ion effects in IL, submitted for publication in an ACS Symposium Series monograph on ionic liquids. To this end, non-toxic organoanions such as acetate and lactate have been used to formulate IL. R. P. Swatlowski, J. D. Holbrey and R. D. Rogers, Green Chem., 2003, 5, 361; L. Crowhurst, N. L. Lancaster, J. M. Perez-Arlandis, and T. Welton, an advance copy of a manuscript dealing with ion effects in IL, submitted for publication in an ACS Symposium Series monograph on ionic liquidS; M. J. Earle, P. B. McCormac and K. R. Seddon, Green Chem., 1999, 1, 23. However, carboxylates are basic, readily engage in hydrogen bonding, and are strongly coordinating towards transition-metal ions. Such attributes are not typical of the fluorous anions on which so many IL compositions are based. Mapped onto an IL, these properties are likely to be useful in some circumstances and detrimental in others.

Bronsted Acid Catalysis

From undergraduate laboratories to chemical manufacturing plants, the use of strong Bronsted acids is ubiquitous. Smith, M. B.; March, J. March's Advanced Organic Chemistry; Wiley-Interscience: New York, 2001; Chapter 8. In this context, solid acids are being more widely used since, as non-volatile materials, they are deemed less noxious than traditional liquid acids. Ritter, S. K. Chem. Eng. News, 2001, 79 (40), 63-67. However, solid acids have shortcomings. Among the more troublesome of these are restricted accessibility of the matrix-bound acidic sites, high mw/active site ratios, and rapid deactivation from coking. Ishihara, K.; Hasegama, A. and Yamamoto, H. Angew. Chem. Int. Ed., 2001, 40, 4077-4079; and Harmer, M. A. and Sun, Q. Appl. Catal. A: General, 2001, 221, 45-62.

Bearing in mind both the advantages and disadvantages of solid acids, the search continues for systems that are Bronsted acids with solid-like non-volatility but that manifest the motility, greater effective surface area and potential activity of a liquid phase. Combining just these characteristics, ionic liquids (IL) have been described as one of the most promising new reaction mediums. Seddon, K. R. J. Chem. Technol. Biotechnol. 1997, 68, 351-356. Not only can these unusual materials dissolve many organic and inorganic substrates, they are also readily recycled and are tunable to specific chemical tasks. Bates, E. D.; Mayton, R. D.; Ntai, I. and Davis, J. H. Jr. J. Am. Chem Soc. 2002, 124, 926-927; Visser, A. E.; Holbrey, J. D.; Rogers, R. D. Chem. Commun., 2001, 2484-2485; Visser, A. E.; Swatloski, R. P.; Reichert, W. M.; Mayton, R.; Sheff, S.; Wierzbicki, A.; Davis, J. H. Jr.; Rogers. R. D. Chem. Commun., 2001, 135-136; Merrigan, T. L.; Bates, E. D.; Dorman; S. C.; Davis, J. H. Jr. Chem. Commun. 2000, 2051-2052; Forrester, K. J.; Davis, J. H. Jr. Tetrahedron Lett., 1999, 40, 1621-1622; and Morrison, D. W.; Forbes D. C.; Davis, J. H. Jr. Tetrahedron Letters, 2001, 42, 6053-6057.

Further, the chemical industry is under significant pressure to replace the volatile organic compounds that are currently used as solvents in organic synthesis. Many of these solvents, such as chlorinated hydrocarbons, are toxic and hazardous for the environment, due to their emissions in the atmosphere and the contamination of aqueous effluents. Ionic liquids (IL) seem to offer a solution to this problem, too. Ionic liquids have no measurable vapor pressure. This means that they don't evaporate, and therefore they emit no hazardous vapors in the atmosphere, and replenishing of the solvent is not required. This property also allows easy separation of volatile products. ILs are able to dissolve a wide range of organic, inorganic and organometallic compounds. Notably, their properties can be adjusted by altering the cation or anion of the IL, allowing for fine tuning of the reaction.

Moreover, many organic transformations, such as Fischer esterification, alcohol dehydrodimerization and the pinacol/benzopinacol rearrangement, require an acidic catalyst. Solid acids are now being used, because, as nonvolatile compounds, they are less hazardous than traditional liquid acids. As noted above, although they are less hazardous, solid acids have several disadvantages, such as restricted accessibility of the matrix-bound acidic sites, high molecular weight/active-site ratios, and rapid deactivation from coking. Cole, A. C.; Jensen, J. L.; Ntai, I.; Tran, K. L. T.; Weaver, K. J.; Forbes, D. C.; Davis, J. H., Jr. J. Am. Chem. Soc. 2002, 124, 5962-5963.

Purification of Gas Mixtures

There is little doubt that petroleum, coal and natural gas will continue to be the primary global fuel and chemical feedstock sources for some years to come. Mills, Mark P. Energy Policy in the Electron Age, Mills-McCarthy & Associates, Inc. <http://www.fossilfuels.org/electric/electron.htm>. Natural gas is regarded as the cleanest of these materials, and as such is being consumed at an accelerating pace. Despite its reputation as a clean fuel, natural gas is usually contaminated with a variety of undesirable materials, especially CO₂ and H₂S. While this level of contamination is very low in gas from certain sources (sweet gas), it is much higher in gas from others (sour gas). As sweet gas reserves are depleted, pressures will build for the increased utilization of sour gas. Oil and Gas R&D Programs: Securing the U.S. Energy, Environmental and Economic Future. Office of Fossil Energy, U.S. Dept. of Energy, Office of Natural Gas and Petroleum Technology: Washington, D.C., 1997. Since admixed CO₂ lowers the fuel value of natural gas, the large amount of it present in sour gas compels its removal prior to combustion. The lower fuel value for sour gas, coupled with the connection between CO₂ and global warming, makes CO₂ capture a commercially important and environmentally desirable process.

One of the most attractive approaches for the separation of a target compound from a mixture of gases in a gas stream is selective absorption into a liquid. Astarita, G,; Savage, D. W.; Bisio, A. Gas Treating with Chemical Solvents; Wiley-Interscience: New York, 1983. Such interactions between gases and pure liquids or solutions are the bases for numerous gas separation technologies, including commercial systems for the removal of CO₂ from natural gas. These scrubbing processes include ones in which the simple, differential dissolution of the target gas into the liquid phase is of principal importance. More common are processes in which a chemical reaction of the target gas with a solute in the liquid phase is the main mode of sequestration. With either mode of gas removal, the vapor pressure of the solvent itself plays a significant role in gas-liquid processes, usually to their detriment. In the case of large-scale CO₂ capture, aqueous amines are used to trap chemically the CO₂ by way of ammonium carbamate formation. In these systems, the uptake of water into the gas stream is particularly problematic. Compounding the water uptake difficulty is the loss into the gas stream of the volatile amine sequestering agent.

A liquid that could facilitate the sequestration of gases without concurrent loss of the capture agent or solvent into the gas stream should prove to be a superior material in such applications. To this end, ionic liquids (low temperature molten salts) have been proposed as solvent-reagents for gas separations. Pez, G. P.; Carlin, R. T.; Laciak, D. V.; Sorensen, J. C. U.S. Pat. No. 4,761,164. Due to the coulombic attraction between the ions of these liquids, they exhibit no measurable vapor pressure up to their thermal decomposition point, generally >300° C. This lack of vapor pressure makes these materials highly attractive for gas processing. Indeed, for these purposes they may be thought of as “liquid solids,” incorporating some of the most useful physical properties of both phases.

Despite the general promise of ionic liquids (IL) in gas treatment, the molten salts used thus far for CO₂ separation are generally “off the shelf” materials, such as (CH₃)₄NF tetrahydrate, that are not optimized for this purpose, frequently depending upon another volatile reagent, water. Pez, G. P.; Carlin, R. T.; Laciak, D. V.; Sorensen, J. C. U.S. Pat. No. 4,761,164; Quinn, R.; Pez, G P. U.S. Pat. No. 4,973,456; and Quinn, R.; Appleby, J. B.; Pez, G. P. J. Am. Chem. Soc., 1995, 117, 329. For instance, the latter salt uses the very weakly basic bifluoride ion to drive the net generation of bicarbonate from CO₂ and water.

The prospects for preparing a broad array of ionic liquids with ions incorporating functional groups are good. Freemantle, M. Chemical & Engineering News, May 15, 2000, 37. Moreover, certain of these new “task-specific” ionic liquids have proven useful in both synthetic and separations applications. Visser, A. E.; Holbrey, J. D.; Rogers, R. D. Chem. Commun., 2001, 2484; Visser, A. E.; Swatloski, R. P.; Reichert, W. M.; Mayton, R.; Sheff, S.; Wierzbicki, A.; Davis, J. H. Jr.; Rogers. R. D. Chem. Commun., 2001, 135; Merrigan, T. L.; Bates, E. D.; Dorman; S. C.; Davis, J. H. Jr. Chem. Commun. 2000, 2051; Fraga-Dubreuil, J.; Bazureau J. P. Tetrahedron Lett., 2001, 42, 6097; and Forrester, K. J.; Davis, J. H. Jr. Tetrahedron Lett., 1999, 40, 1621.

### SUMMARY OF THE INVENTION

In one aspect, the present invention relates to a new class of salts that form ionic liquids at relatively low temperatures. In some examples the salts are liquid at about room temperature. The new class of ionic liquid salts comprises anions known to the sweetener industry and an onium cation. In certain embodiments, the sweetener anion is saccharinate (Sac), acesulfamate (Ace), or cyclamate (Cyc), or derivatives thereof. The onium cation may comprise a wide array of cations where the positive charge results from exceeding an element's standard bond number. Ionic liquids have been used with promising results in such areas as non-volatile reaction mediums for a host of chemical reactions, separations in the gas phase, altering dissolution rates, and as heat storage media.

In another aspect, the present invention relates to a method of removing carbon dioxide, carbonyl sulfide, sulfur dioxide, sulfur trioxide, hydrogen sulfide or a carbonyl-containing compound from a gaseous or liquid mixture, comprising the step of exposing a gaseous or liquid mixture to a salt of the present invention.

In another aspect, the present invention relates to a method of transporting carbon dioxide, carbonyl sulfide, sulfur dioxide, sulfur trioxide, hydrogen sulfide or a carbonyl-containing compound from a first gaseous or liquid mixture to a second gaseous or liquid mixture, comprising the step of exposing a first gaseous or liquid mixture to a salt of the present invention; and subsequently exposing the salt of the present invention to a second gaseous or liquid mixture, thereby transporting carbon dioxide, carbonyl sulfide, sulfur dioxide, sulfur trioxide, hydrogen sulfide or a carbonyl-containing compound to the second gaseous or liquid mixture.

In another aspect, the present invention relates to a method of removing an alkene, alkyne or carbon monoxide from a mixture, comprising the step of exposing a mixture to a complex formed from a transition metal and a salt of the present invention.

In another aspect, the present invention relates to a method of catalyzing an acid-catalyzed chemical reaction to give a product, comprising the step of exposing a reactant mixture to a salt of the present invention.

In another aspect, the present invention relates to a method of catalyzing an based-catalyzed chemical reaction to give a product, comprising the step of exposing a reactant mixture to a salt of the present invention.

In another aspect, the present invention relates to a method of preparing a solution, comprising the step of combining a solute and a solvent to produce a solution wherein said solvent is a salt of the present invention.

In another aspect, the present invention relates to a heat storage medium comprising a salt of the present invention.

These embodiments of the present invention, other embodiments, and their features and characteristics, will be apparent from the description, drawings and claims that follow.

### BRIEF DESCRIPTION OF THE FIGURES

FIG. 1 depicts the structures of saccharinate (Sac), acesulfamate (Ace), cyclamate (Cyc), TSAC, and bis(trifluoromethyl)sulfonyl imide (Tf₂N⁻).

FIG. 2 depicts the ¹H NMR of imidazolium C²—H as a probe of anion similarities.

FIG. 3 depicts the IR spectra of CH₂Cl₂ solutions initially 1.60 mM in {Rh}X and 3.20 mM in 4-picolone, showing the relative anion affinity of X⁻ for {Rh}⁺ via partitioning into {Rh}X and [{Rh}4pic⁺]X⁻.

### DETAILED DESCRIPTION OF THE INVENTION

The invention will now be described more fully with reference to the accompanying examples, in which certain preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The term “ionic liquid” or “IL” as used herein means an organic salt or hydrate thereof with a melting point less than about 150° C. In a preferred embodiment, the ionic liquid has a melting point of less than about 100° C. In a particularly preferred embodiment, the ionic liquid has a melting point of less than about room temperature.

The term “onium cation” as used herein means any cation derived by the addition of a chemical group to the nitrogen, chalcogen, and halogen familes of elements where the standard bonding number has been exceeded.

The term “heteroatom” as used herein means an atom of any element other than carbon or hydrogen. Preferred heteroatoms are boron, nitrogen, oxygen, phosphorus, sulfur and selenium.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, J. March, Advanced Organic Chemistry, McGraw Hill Book Company, New York, (1977 edition) pp. 251-259. The Hammett constant values are generally negative for electron donating groups (σ[P]=−0.66 for NH₂) and positive for electron withdrawing groups (σ[P]=0.78 for a nitro group), σ[P] indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formula: 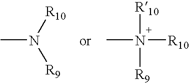
wherein R₉, R₁₀ and R′₁₀ each independently represent a group permitted by the rules of valence.

The term “acylamino” is art-recognized and refers to a moiety that can be represented by the general formula: 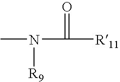
wherein R₉ is as defined above, and R′₁₁ represents a hydrogen, an alkyl, an alkenyl or —(CH₂)ₘ—R₈, where m and R₈ are as defined above.

The term “amido” is art recognized as an amino-substituted carbonyl and includes a moiety that can be represented by the general formula: 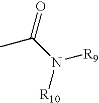
wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethyl thio, and the like.

The term “carbonyl” is art recognized and includes such moieties as can be represented by the general formula: 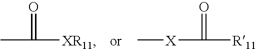
wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R₁₁ 1 is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “sulfonate” is art recognized and includes a moiety that can be represented by the general formula: 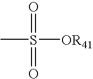
in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is art recognized and includes a moiety that can be represented by the general formula: 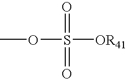
in which R₄₁ is as defined above.

The term “sulfonylamino” is art recognized and includes a moiety that can be represented by the general formula:

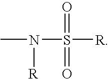

The term “sulfamoyl” is art-recognized and includes a moiety that can be represented by the general formula:

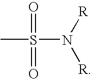

The term “sulfonyl”, as used herein, refers to a moiety that can be represented by the general formula: 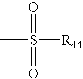
in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula: 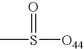
in which R₄₄ is selected from the group consisting of hydrogen, alkyl, alkenyl, alkynyl, cycloalkyl, heterocyclyl, aralkyl, or aryl.

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R₇, m and R₇ being defined above.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers. Moreover, the enantiomers of a racemic mixture may be separated using chiral chromatography, e.g., chiral HPLC.

For purposes of this invention, the chemical elements are identified in accordance with the Periodic Table of the Elements, CAS version, Handbook of Chemistry and Physics, 67th Ed., 1986-87, inside cover.

Overview

One aspect of the present invention relates to the use of saccharinate (Sac), acesulfamate (Ace), cyclamate (Cyc), and derivatives thereof, in the formation of new ionic liquids. As their alkali-metal salts, the anions are in widespread use in foodstuffs as non-nutrititive sweeteners. G. R. von Rymon Lipinski. Ullman's Encyclopedia of Industrial Chemistry, 6^{th} Edition; Wiley-VCH: Weinheim, 2003; Vol. 35, p. 407. Significantly, when incorporated into IL, these anions exhibit behaviors that in several respects more closely resemble those of certain fluorous anions than those of common carboxylates.

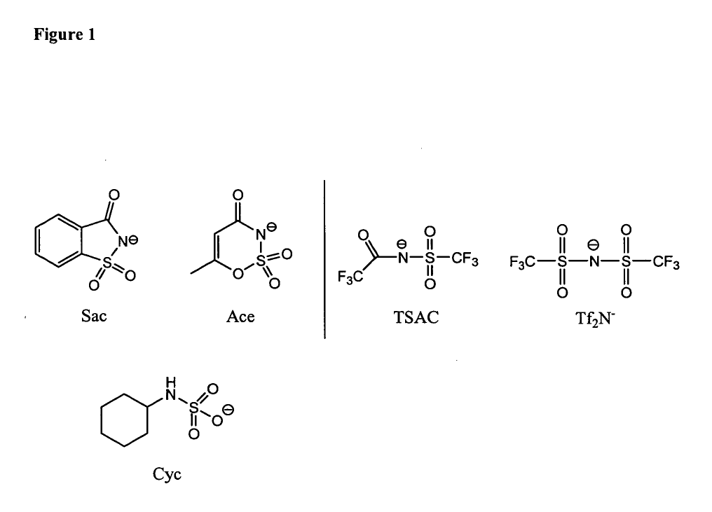

Both of the sweeteners Sac and Ace are N-acyl-N-sulfonyl imides (FIG. 1). As such, they bear a similarity to the TSAC anion introduced by Matsumoto as a substitute for bis(trifluoromethyl)sulfonyl imide (Tf₂N⁻) in IL chemistry. H. Matsumoto, H. Kageyama and Y. Miyazaki, Chem. Commun., 2002. Notably, the Sac and Ace anions are, unlike TSAC and Tf₂N⁻, non-fluorous and have well established toxicological profiles. G.-R. von Rymon Lipinski. Ullman's Encyclopedia of Industrial Chemistry, 6^{th} Edition; Wiley-VCH: Weinheim, 2003; Vol. 35, p. 407.

When combined with cations, such as onium cations, each anion gives rise to salts melting below 100° C. Prototypes have been prepared in which each anion is paired with an array of cations, and select examples are presented in Table 1. All of the new ionic liquids are prepared via metathesis, combining the requisite cation halide salt with the sodium, potassium, or silver salt of the sweetener in acetone or acetone/water. The resulting IL are miscible with polar organic solvents, such as acetone, acetonitrile, and methanol, and insoluble in less polar species, such as ether, n-hexane and toluene. Conversely, the latter are generally soluble to a fair degree in the ionic liquids. Like IL incorporating small fluorous organoanions (e.g. CF₃CO₂⁻, CF₃SO₃⁻), the Sac, Ace, and Cyc salts are water miscible.

TABLE 1
Select properties of representative saccharin and
acesulfame-derived ionic liquids.

| Ionic Liquid^{a} | H-C² δ^{b} | mp (° C.)^{c} |
|---|---|---|
| [BMIM]Sac | 9.53 | RT |
| [PMIM]Sac | 9.60 | 64 |
| [HMIM]Sac | 9.80 | RT |
| [NMIM]Sac | 9.64 | Rt |
| [BDMIM]Sac^{d} | — | 70 |
| [BMIM]Ace | 9.51 | RT |
| [HMIM]Ace | 9.62 | RT |
| [NMIM]Ace | 9.59 | RT |
| [Me₃N(CH₂)₂OC(O)(CH₂)₂CH₃]Sac | — | 89 |
| [P1(CH₂)₂OCH₂CH₃]Sac^{c} | — | RT |
| [n-Pr₄N]Sac | — | 96 |
| [MeEt₃N]Sac | — | 26^{f} |
| [Me₃N(CH₂)₂OC(O)(CH₂)₂CH₃]Ace | — | 49 |
| [n-Pr₄N]Ace | — | 73 |
| [MeEt₃N]Ace | — | 39^{f} |

^{a}[BMIM] = 1-methyl-3-(n-butyl)imidazolium; [HMIM] = 1-methyl-3-(n-hexyl)imidazolium; [NMIM] = 1-methyl-3-(n-nonyl) imidazolium.
^{b}Imidazolium ring C² proton, 300 MHz, 0.50 M, acetone-d⁶, δ.
^{c}RT = liquid at or below room temperature. These compounds have remained liquids despite repeated attempts to crystallize them.
^{d}[BDMIM] = 1-butyl-2,3-dimethyl imidazolium.
^{e}P1 = N-methylpyrrolidinyl. D. R. MacFarlane, P. Meakin, J. Sun, N. Amini and M. Forsyth, J. Phys. Chem., 1999, 103, 4164.
^{f}These materials are waxy semi-solids at room temperature. The reported mp is the temperature at which the sample fully liquified.

Several of the new IL are room-temperature liquidS; they are somewhat viscous, but become visibly less so with modest heating or when small quantities of water or an organic solvent is added. The IL with melting points above room temperature readily form long-lived supercooled phases. In terms of melting points and viscosities, IL derived from the sweetener anions generally resemble those derived from other non-fluorous organoanions, as well as those of some larger perfluoro anions, such as nonafluorobutanesulfonate, NfO⁻.

NMR and IR Spectra

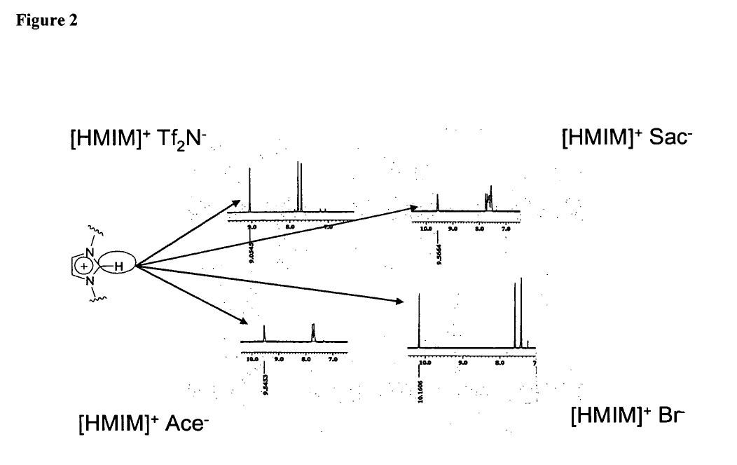

The new ionic liquids have been characterized by ¹H- and ¹³C-NMR as well as FAB-MS. The ¹H-NMR spectra of the imidazolium-Sac and Ace IL are particularly informative when viewed in the context of same-cation salts of other anions. It has been shown that for solutions of like concentration, a correlation exists between the chemical shift of the ring protons (especially that on C²) and the hydrogen-bonding capacity of the anion. (a) P. Bonhote, A.-P. Dias, N. Papageorgiou, K. Kalyanasundaram and M. Gratzel, Inorg. Chem., 35, 1996, 1168. (b) A. Elaiwi, P. B. Hitchcock, K. R. Seddon, N. Srinivasan, Y. M. Tan, T. Welton and J. A. Zora, J. Chem. Soc., Dalton Trans., 1995, 3467. (c) A. G. Avent, P. A. Chalconer, M. P. Day, K. R. Seddon and T. Welton, J. Chem. Soc., Dalton Trans., 1994, 3405. Generally, the H—C² resonance is observed at higher δ values with more strongly H-bonding species (halides, carboxylates, etc.) and at lower values when more poorly H-bonding anions (PF₆⁻, Tf₂N⁻, etc.) are used. Comparatively, the sweetener anions of the IL produce H—C² shifts, see Table 1 and FIG. 2, falling between those observed for IL with perfluorocarboxylate and perfluorosufonate anions. (a) P. Bonhote, A.-P. Dias, N. Papageorgiou, K. Kalyanasundaram and M. Gratzel, Inorg. Chem., 35, 1996, 1168. (b) A. Elaiwi, P. B. Hitchcock, K. R. Seddon, N. Srinivasan, Y. M. Tan, T. Welton and J. A. Zora, J. Chem. Soc., Dalton Trans., 1995, 3467. (c) A. G. Avent, P. A. Chalconer, M. P. Day, K. R. Seddon and T. Welton, J. Chem. Soc., Dalton Trans., 1994, 3405. For example, the H—C² proton in [BMIM]Sac appears at 9.53 ppm and that in [BMIM]Ace at 9.51 ppm. The trifluoroacetate salt of the same cation exhibits the H—C² signal at δ 9.88. When paired with either NfO⁻ or TfO⁻, the same resonance is observed at δ 9.11. In the only reported imidazolium TSAC salt 1-methyl-3-(ethyl)imidazolium ([EMIM]; acetone-d⁶), the signal is observed at δ 9.06. H. Matsumoto, H. Kageyama and Y. Miyazaki, Chem. Commun., 2002. In striking contrast to these values, the H—C² signal of [BMIM]Cl is observed at δ 10.79 and that for [BMIM]acetate at 11.02. P. Bonhote, A.-P. Dias, N. Papageorgiou, K. Kalyanasundaram and M. Gratzel, Inorg. Chem., 35, 1996, 1168. The chemical shifts of the H—C² resonances of the Sac and Ace salts acquired at 0.50 M concentration in CDCl₃ gives peaks 0.15-0.20 ppm downfield relative to their positions in acetone. This is likely indicative of the formation in this solvent of “quasi-molecular” species that pi-stack, shielding the C² protons. A. G. Avent, P. A. Chalconer, M. P. Day, K. R. Seddon and T. Welton, J. Chem. Soc., Dalton Trans., 1994, 3405. In either solvent, the chemical shifts of the C²—protons in both the Sac⁻ and Ace⁻ salts exhibit only a small change as a function of concentration. Like the C²—H chemical shift values, this phenomenon is strongly indicative of a weak capacity on the part of Sac⁻ and Ace⁻ to engage in hydrogen bonds in solution with the imidazolium cations. (a) P. Bonhote, A.-P. Dias, N. Papageorgiou, K. Kalyanasundaram and M. Gratzel, Inorg. Chem., 35, 1996, 1168. (b) A. Elaiwi, P. B. Hitchcock, K. R. Seddon, N. Srinivasan, Y. M. Tan, T. Welton and J. A. Zora, J. Chem. Soc., Dalton Trans., 1995, 3467. (c) A. G. Avent, P. A. Chalconer, M. P. Day, K. R. Seddon and T. Welton, J. Chem. Soc., Dalton Trans., 1994, 3405.

Given the low H-bonding capacity suggested by the NMR data for these non-fluorous anions, we next assessed their relative transition-metal coordinating ability. Previous work has shown that v_{Rh-CO} in trans-Rh(PPh₃)₂(CO)X (Vaska complexeS; {Rh}X hereafter) is sensitive to the donor nature of ligand X⁻, increasing as bonding power decreases.¹⁰ The values for v_{Rh-CO} for a range of weakly binding X⁻ and relative affinities thereof for {Rh}⁺ have been determined.¹¹ Both AgSac and AgAce cleanly abstracted Cl⁻ from {Rh}Cl in benzene or CDCl₃ and transferred the respective sweetener anion to {Rh}⁺; yellow microcrystals of the new species were isolated in over 90% yield. Like the new ILs, these complexes have been fully characterized by spectroscopic means, and satisfactory elemental analyses have been obtained as well. Transition-metal complexes of sweetener anions have been documented. Hughes, M. N., “Coordination Compounds in Biology” in Comprehensive Coordination Chemistry, G. Wilkinson, Ed., Pergamon: Oxford, 1987, vol. 6, Chapter 62.1, p. 541. However, the v_{Rh-CO} of 1987 cm⁻¹ for {Rh} Sac and 1980 cm⁻¹ for {Rh}Ace did not match their respective affinities for {Rh}⁺ as determined in CDCl₃ by ¹⁹F and/or ³¹P NMR for (below) eq. 1 (Z=TFA, K=0.091; Z=3-FC₆H₄CO₂, K=31; Z=CH₃CO₂, K=99;) and eq. 2 (Z=TFA, K=1.1).
{Rh}Sac+[N(PPh₃)₂]Z={Rh}Z+[N(PPh₃)₂]Sac (1)
{Rh}Ace+[N(PPh₃)₂]Z={Rh}Z+[N(PPh₃)₂]Ace (2)

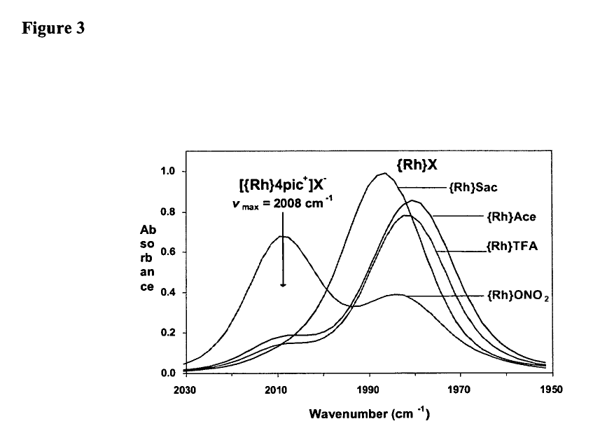

The actual order of relative affinities for {Rh}⁺ of Sac⁻>TFA⁻>Ace⁻>ONO₂⁻ are displayed in FIG. 3 by their increasing tendancy to be displaced from their complexes by 4-picoline. Thus Sac⁻ behaves not like the quite weakly binding OPOF₂⁻ (same values for v_{Rh-CO}) but more akin the mid-range 3-FC₆H₄CO₂—, and Ace⁻ (same v_{Rh-CO} as for Cl⁻) just slightly weaker than the moderately weakly binding TFA⁻. While the anion affinities for {Rh}⁺ fail to closely correlate with ³¹P and ¹³C NMR parameters of {Rh}X, it is clear from the competition reactions that the sweetener anions complex {Rh}⁺ much more weakly than does acetate.

Compounds of the Invention

The compounds of the present invention may be prepared via metathesis reactions which allow for easy preparation of a wide variety of IL salts with different cations and anions. In general, an alkali metal salt of the anionic sweetener is reacted with a halide salt of the onium cation to yield the salt of the ionic liquid. In another variation, a silver salt of the anionic sweetener may be used to give the ionic liquid salt and a silver halide in high yields. The synthetic scheme is outlined below in Scheme 1 for Sac and Ace anions.

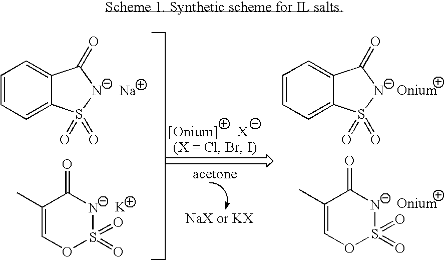

In certain embodiments, the present invention relates to a salt represented by formula I:
C⁺A⁻ I
wherein:
C⁺ represents an onium cation, and
A⁻ represents an anion of formula Ia: 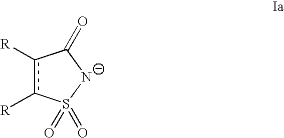
   wherein, independently for each occurrence:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈; or the two R taken together represent a substituted or unsubstituted fused cycloalkyl, cycloalkenyl, aryl, or heteroaryl ring;
   R₈ represents cycloalkyl, aryl, or heteroaryl;
   n represents an integer from 1-10 inclusive; and 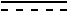
   represents a single or double bond; or
   an anion of formula Ib: 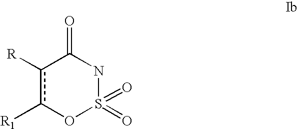
   wherein:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
   R₁ represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
   or R and R₁ taken together represent a substituted or unsubstituted fused cycloalkyl, cycloalkenyl, aryl, or heteroaryl ring;
   R₈ represents cycloalkyl, aryl, or heteroaryl;
   n represents an integer from 1-10 inclusive; and 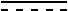
   represents a single or double bond; or
   an anion of formula Ic: 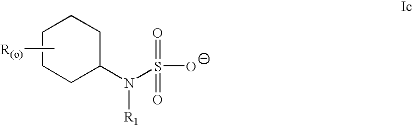
      wherein, independently for each occurrence:

R is halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈, or any two adjacent R taken together represent a substituted or unsubstituted fused cycloalkyl, cycloalkenyl, aryl, or heteroaryl ring;
R₁ is H, halide, alkyl, alkenyl, alkynyl, cycloalky, aryl, heteroaryl, aralkyl, heteroaralkyl, hydroxy, amino, alkoxy, carboxyl, keto, aldehyde, or —(CH₂)ₙ—R₈;
R₈ represents cycloalkyl, aryl, or heteroaryl;
n represents an integer from 1-10 inclusive; and
o represents an integer from 0 to 11 inclusive.

In certain embodiments, the present invention relates to a salt represented by formula I and the attendant definitions, wherein A⁻ is an anion of formula Ia.

In certain embodiments, the present invention relates to a salt represented by formula I and the attendant definitions, wherein A⁻ is an anion of formula Ia and 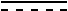
represents a double bond.

In certain embodiments, the present invention relates to a salt represented by formula I and the attendant definitions, wherein A⁻ is an anion of formula Ia and the two R groups taken together represent a fused aryl ring.

In certain embodiments, the present invention relates to a salt represented by formula I and the attendant definitions, wherein A⁻ is an anion of formula Ia and the two R groups taken together represent a fused benzene ring.

In certain embodiments, the present invention relates to a salt represented by formula I and the attendant definitions, wherein A⁻ is an anion of formula Ib.

In certain embodiments, the present invention relates to a salt represented by formula I and the attendant definitions, wherein A⁻ is an anion of formula Ib and 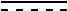
represents a double bond.

In certain embodiments, the present invention relates to a salt represented by formula I and the attendant definitions, wherein A⁻ is an anion of formula Ib and R represents H.

In certain embodiments, the present invention relates to a salt represented by formula I and the attendant definitions, wherein A⁻ is an anion of formula Ib and R₁ represents methyl.

In certain embodiments, the present invention relates to a salt represented by formula I and the attendant definitions, wherein A⁻ is an anion of formula Ib, wherein 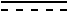
represents a double bond and R represents H.

In certain embodiments, the present invention relates to a salt represented by formula I and the attendant definitions, wherein A⁻ is an anion of formula Ib, wherein 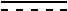
represents a double bond, R represents H, and R₁ represents methyl.

In certain embodiments, the present invention relates to a salt represented by formula I and the attendant definitions, wherein A⁻ is an anion of formula Ic.

In certain embodiments, the present invention relates to a salt represented by formula I and the attendant definitions, wherein A⁻ is an anion of formula Ic and o is 0.

In certain embodiments, the present invention relates to a salt represented by formula I and the attendant definitions, wherein A⁻ is an anion of formula Ic and R₁ is H.

In certain embodiments, the present invention relates to a salt represented by formula I and the attendant definitions, wherein A⁻ is an anion of formula Ic, wherein o is 0 and R₁ is H.

In certain embodiments, the present invention relates to a salt represented by formula I and the attendant definitions, wherein the onium cation is selected from the following: a phosphonium, ammonium, imidazonium, pyrrolidinium, pyridinium, thiazolium, arsonium, stibonium, oxonium, sulfonium, selenonium, telluronium, fluoronium, chloronium, bromonium, or iodonium cation.

In certain embodiments, the present invention relates to a salt represented by formula I and the attendant definitions, wherein the onium cation is an ammonium, imidazonium, phosphonium, pyridinium, or thiazolium cation.

In certain embodiments, the present invention relates to a salt represented by formula I and the attendant definitions, wherein the onium cation is represented by formula Id: 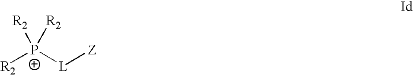
wherein, independently for each occurrence:

R₂ represents alkyl, fluoroalkyl, cycloalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —CO₂R₂, —C(O)N(R″)₂, —C(O)N(R″)N(R″)₂, —N(R′)₂, —OR′, —SR′, —S(O)R″, —S(O)₂R″, —CN, —N(R″)P(O)(R)₂, —C(OR′)(R″)₂, alkenyl, or alkynyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
Ar represents aryl or heteroaryl;
J represents O, S, NR′, cycloalkyl, or heterocyclyl;
R₈ represents cycloalkyl, aryl, or heteroaryl;
m represents an integer from 1-10 inclusive; and
n represents an integer in the range 1-10 inclusive.

In certain embodiments, the present invention relates to a salt represented by formula I and the attendant definitions, wherein the onium cation is represented by formula Id and R₂ represents alkyl.

In certain embodiments, the present invention relates to a salt represented by formula I and the attendant definitions, wherein the onium cation is represented by formula Id and Z represents H.

In certain embodiments, the present invention relates to a salt represented by formula I and the attendant definitions, wherein the onium cation is represented by formula Id and L represents (C(R₃)₂)ₙ.

In certain embodiments, the present invention relates to a salt represented by formula I and the attendant definitions, wherein the onium cation is represented by formula Id, and wherein A⁻ represents an anion of formula Ia.

In certain embodiments, the present invention relates to a salt represented by formula I and the attendant definitions, wherein the onium cation is represented by formula Id, and wherein A⁻ represents an anion of formula Ib.

In certain embodiments, the present invention relates to a salt represented by formula I and the attendant definitions, wherein the onium cation is represented by formula Id, and wherein A⁻ represents an anion of formula Ic.

In certain embodiments, the present invention relates to a salt represented by formula I and the attendant definitions, wherein the onium cation is represented by formula Id, wherein R₂ represents alkyl; and Z represents H.

In certain embodiments, the present invention relates to a salt represented by formula I and the attendant definitions, wherein the onium cation is represented by formula Id, wherein R₂ represents alkyl; Z represents H; and L represents (C(R₃)₂)ₙ.

In certain embodiments, the present invention relates to a salt represented by formula I and the attendant definitions, wherein the onium cation is represented by formula Id, wherein R₂ represents alkyl; Z represents H; L represents (C(R₃)₂)ₙ; and A⁻ represents an anion of formula Ia.

In certain embodiments, the present invention relates to a salt represented by formula I and the attendant definitions, wherein the onium cation is represented by formula Id, wherein R₂ represents alkyl; Z represents H; L represents (C(R₃)₂)ₙ; and A⁻ represents an anion of formula Ib.

In certain embodiments, the present invention relates to a salt represented by formula I and the attendant definitions, wherein the onium cation is represented by formula Id, wherein R₂ represents alkyl; Z represents H; L represents (C(R₃)₂)ₙ; and A⁻ represents an anion of formula Ic.

In certain embodiments, the present invention relates to a salt represented by formula I and the attendant definitions, wherein the onium cation is represented by formula Id, wherein R₂ represents alkyl; Z represents H; L represents (C(R₃)₂)ₙ; and A⁻ represents an anion of formula Ia, wherein the two R groups taken together represent a fused aryl ring.

In certain embodiments, the present invention relates to a salt represented by formula I and the attendant definitions, wherein the onium cation is represented by formula Id, wherein R₂ represents alkyl; Z represents H; L represents (C(R₃)₂)ₙ; and A⁻ represents an anion of formula Ia, wherein the two R groups taken together represent a fused benzene ring.

In certain embodiments, the present invention relates to a salt represented by formula I and the attendant definitions, wherein the onium cation is represented by formula Id, wherein R₂ represents alkyl; Z represents H; L represents (C(R₃)₂)ₙ; and A⁻ represents an anion of formula Ib, wherein 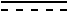
represents a double bond.

In certain embodiments, the present invention relates to a salt represented by formula I and the attendant definitions, wherein the onium cation is represented by formula Id, wherein R₂ represents alkyl; Z represents H; L represents (C(R₃)₂)ₙ; and A⁻ represents an anion of formula Ib, wherein 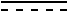
represents a double bond, and R represents H.

In certain embodiments, the present invention relates to a salt represented by formula I and the attendant definitions, wherein the onium cation is represented by formula Id, wherein R₂ represents alkyl; Z represents H; L represents (C(R₃)₂)ₙ; and A⁻ represents an anion of formula Ib, wherein 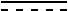
represents a double bond, R represents H, and R₁ represents methyl.

In certain embodiments, the present invention relates to a salt represented by formula I and the attendant definitions, wherein the onium cation is represented by formula Id, wherein R₂ represents alkyl; Z represents H; L represents (C(R₃)₂)ₙ; and A⁻ represents an anion of formula Ic, wherein o is 0.

In certain embodiments, the present invention relates to a salt represented by formula I and the attendant definitions, wherein the onium cation is represented by formula Id, wherein R₂ represents alkyl; Z represents H; L represents (C(R₃)₂)ₙ; and A⁻ represents an anion of formula Ic, wherein o is 0, and R₁ is H.

In certain embodiments, the present invention relates to a salt represented by formula I and the attendant definitions, wherein the onium cation is represented by formula Id, wherein R₂ represents n-butyl; Z represents H; L represents —(CH₂)₄—; and A⁻ represents an anion of formula Ia, wherein the two R groups taken together represent a fused benzene ring.

In certain embodiments, the present invention relates to a salt represented by formula I and the attendant definitions, wherein the onium cation is represented by formula Ie: 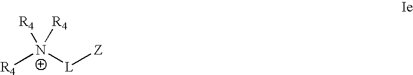
wherein, independently for each occurrence:

R₄ represents alkyl, fluoroalkyl, cycloalkyl, aryl, heteroaryl, aralkyl, heteroaralkyl, or —(CH₂)ₙ—R₈; or ⁺N(R₄)₃ taken together represents pyridinium, imidazolium, benzimidazolium, pyrazolium, benzpyrazolium, indazolium, thiazolium, benzthiazolium, oxazolium, benzoxazolium, isoxazolium, isothiazolium, imdazolidenium, guanidinium, quinuclidinium, triazolium, tetrazolium, quinolinium, isoquinolinium, piperidinium, pyrrolidinium, morpholinium, pyridazinium, pyrazinium, piperazinium, triazinium, azepinium, or diazepinium;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —OC(O)R′, —CO₂R₄, —C(O)N(R″)₂, —C(O)N(R″)N(R″)₂, —N(R′)₂, —OR′, —SR′, —S(O)R″, —S(O)₂R″, —CN, —N(R″)P(O)(R₄)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 1-10 inclusive.

In certain embodiments, the present invention relates to a salt represented by formula I and the attendant definitions, wherein the onium cation is represented by formula Ie, wherein R₄ represents independently for each occurrence alkyl or aryl.

In certain embodiments, the present invention relates to a salt represented by formula I and the attendant definitions, wherein the onium cation is represented by formula Ie, wherein Z represents H or —OC(O)R′.

In certain embodiments, the present invention relates to a salt represented by formula I and the attendant definitions, wherein the onium cation is represented by formula Ie, wherein L represents (C(R₃)₂)ₙ.

In certain embodiments, the present invention relates to a salt represented by formula I and the attendant definitions, wherein the onium cation is represented by formula Ie, wherein A⁻ represents an anion of formula Ia.

In certain embodiments, the present invention relates to a salt represented by formula I and the attendant definitions, wherein the onium cation is represented by formula Ie, wherein A⁻ represents an anion of formula Ib.

In certain embodiments, the present invention relates to a salt represented by formula I and the attendant definitions, wherein the onium cation is represented by formula Ie, wherein A⁻ represents an anion of formula Ic.

In certain embodiments, the present invention relates to a salt represented by formula I and the attendant definitions, wherein the onium cation is represented by formula Ie, wherein R₄ represents alkyl or aryl; and Z represents H or —OC(O)R′.

In certain embodiments, the present invention relates to a salt represented by formula I and the attendant definitions, wherein the onium cation is represented by formula Ie, wherein R₄ represents alkyl or aryl; Z represents H or —OC(O)R′; and L represents (C(R₃)₂)ₙ.

In certain embodiments, the present invention relates to a salt represented by formula I and the attendant definitions, wherein the onium cation is represented by formula Ie, wherein R₄ represents alkyl or aryl; Z represents H or —OC(O)R′; L represents (C(R₃)₂)ₙ; and A⁻ represents an anion of formula Ia.

In certain embodiments, the present invention relates to a salt represented by formula I and the attendant definitions, wherein the onium cation is represented by formula Ie, wherein R₄ represents alkyl or aryl; Z represents H or —OC(O)R′; L represents (C(R₃)₂)ₙ; and A⁻ represents an anion of formula Ib.

In certain embodiments, the present invention relates to a salt represented by formula I and the attendant definitions, wherein the onium cation is represented by formula Ie, wherein R₄ represents alkyl or aryl; Z represents H or —OC(O)R′; L represents (C(R₃)₂)ₙ; and A⁻ represents an anion of formula Ic.

In certain embodiments, the present invention relates to a salt represented by formula I and the attendant definitions, wherein the onium cation is represented by formula Ie, wherein R₄ represents alkyl or aryl; Z represents H or —OC(O)R′; L represents (C(R₃)₂)ₙ; and A⁻ represents an anion of formula Ia, wherein the two R groups taken together represent a fused aryl ring.

In certain embodiments, the present invention relates to a salt represented by formula I and the attendant definitions, wherein the onium cation is represented by formula Ie, wherein R₄ represents alkyl or aryl; Z represents H or —OC(O)R′; L represents (C(R₃)₂)ₙ; and A⁻ represents an anion of formula Ia, wherein the two R groups taken together represent a fused benzene ring.

In certain embodiments, the present invention relates to a salt represented by formula I and the attendant definitions, wherein the onium cation is represented by formula Ie, wherein R₄ represents alkyl or aryl; Z represents H or —OC(O)R′; L represents (C(R₃)₂)ₙ; and A⁻ represents an anion of formula Ib, wherein 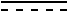
represents a double bond.

In certain embodiments, the present invention relates to a salt represented by formula I and the attendant definitions, wherein the onium cation is represented by formula Ie, wherein R₄ represents alkyl or aryl; Z represents H or —OC(O)R′; L represents (C(R₃)₂)ₙ; and A⁻ represents an anion of formula Ib, wherein 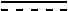
represents a double bond, and R represents H.

In certain embodiments, the present invention relates to a salt represented by formula I and the attendant definitions, wherein the onium cation is represented by formula Ie, wherein R₄ represents alkyl or aryl; Z represents H or —OC(O)R′; L represents (C(R₃)₂)ₙ; and A⁻ represents an anion of formula Ib, wherein 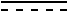
represents a double bond, R represents H, and R₁ represents methyl.

In certain embodiments, the present invention relates to a salt represented by formula I and the attendant definitions, wherein the onium cation is represented by formula Ie, wherein R₄ represents alkyl or aryl; Z represents H or —OC(O)R′; L represents (C(R₃)₂)ₙ; and A⁻ represents an anion of formula Ic, wherein o is 0.

In certain embodiments, the present invention relates to a salt represented by formula I and the attendant definitions, wherein the onium cation is represented by formula Ie, wherein R₄ represents alkyl or aryl; Z represents H or —OC(O)R′; L represents (C(R₃)₂)ₙ; and A⁻ represents an anion of formula Ic, wherein o is 0 and R₁ represents H.

In certain embodiments, the present invention relates to a salt represented by formula I and the attendant definitions, wherein the onium cation is represented by formula Ie, wherein R₄ represents methyl; Z represents —OC(O)CH₂CH₂CH₃; L represents —(CH₂)₂—; and A⁻ represents an anion of formula Ia, wherein the two R groups taken together form a fused benzene ring.

In certain embodiments, the present invention relates to a salt represented by formula I and the attendant definitions, wherein the onium cation is represented by formula Ie, wherein R₄ represents n-propyl; Z represents H; L represents —(CH₂)₃—; and A⁻ represents an anion of formula Ia, wherein the two R groups taken together form a fused benzene ring.

In certain embodiments, the present invention relates to a salt represented by formula I and the attendant definitions, wherein the onium cation is represented by formula Ie, wherein R₄ represents ethyl; Z represents H; L represents —(CH₂)—; and A⁻ represents an anion of formula Ia, wherein the two R groups taken together form a fused benzene ring.

In certain embodiments, the present invention relates to a salt represented by formula I and the attendant definitions, wherein the onium cation is represented by formula Ie, wherein R₄ represents ethyl; Z represents H; L represents —(CH₂)₂—; and A⁻ represents an anion of formula Ia, wherein the two R groups taken together form a fused benzene ring.

In certain embodiments, the present invention relates to a salt represented by formula I and the attendant definitions, wherein the onium cation is represented by formula Ie, wherein R₄ represents methyl; Z represents —OC(O)CH₂CH₂CH₃; L represents —(CH₂)₂—; and A⁻ represents an anion of formula Ib, wherein 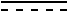
represents a double bond, R represents H, and R₁ represents methyl.

In certain embodiments, the present invention relates to a salt represented by formula I and the attendant definitions, wherein the onium cation is represented by formula Ie, wherein R₄ represents n-propyl; Z represents H; L represents —(CH₂)₃—; and A⁻ represents an anion of formula Ib, wherein 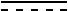
represents a double bond, R represents H, and R₁ represents methyl.

In certain embodiments, the present invention relates to a salt represented by formula I and the attendant definitions, wherein the onium cation is represented by formula Ie, wherein R₄ represents ethyl; Z represents H; L represents —(CH₂)—; and A⁻ represents an anion of formula Ib, wherein 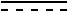
represents a double bond, R represents H, and R₁ represents methyl.

In certain embodiments, the present invention relates to a salt represented by formula I and the attendant definitions, wherein the onium cation is represented by formula Ie, wherein R₄ represents ethyl; Z represents H; L represents —(CH₂)₂—; and A⁻ represents an anion of formula Ib, wherein 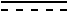
represents a double bond, R represents H, and R₁ represents methyl.

In certain embodiments, the present invention relates to a salt represented by formula I and the attendant definitions, wherein the onium cation is represented by formula Ie, wherein R₄ represents methyl; Z represents —OC(O)CH₂CH₂CH₃; L represents —(CH₂)₂—; and A⁻ represents an anion of formula Ic, wherein o is 0 and R₁ represents H.

In certain embodiments, the present invention relates to a salt represented by formula I and the attendant definitions, wherein the onium cation is represented by formula If: 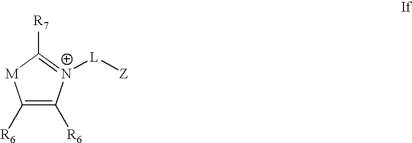
wherein, independently for each occurrence:
M represents NR₅ or S;
R₅ represents H, alkyl, fluoroalkyl, cycloalkyl, aryl, heteroaryl, aralkyl, heteroaralkyl, or —(CH₂)ₙ—R₈; or R₅ is represented by formula If-a: 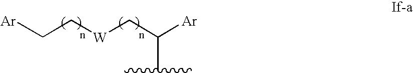
   wherein, independently for each occurrence:
   Ar represents a substituted or unsubstituted aryl or heteroaryl ring; and
   W represents O, NR₇, or S;
R₆ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R₇ represents H, alkyl, fluoroalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —CO₂R₅, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 0-10 inclusive.

In certain embodiments, the present invention relates to a salt represented by formula I and the attendant definitions, wherein the onium cation is represented by formula If, wherein M is NR₅ and R₅ represents alkyl or is represented by formula If-a.

In certain embodiments, the present invention relates to a salt represented by formula I and the attendant definitions, wherein the onium cation is represented by formula If, wherein R₆ represents H or alkyl.

In certain embodiments, the present invention relates to a salt represented by formula I and the attendant definitions, wherein the onium cation is represented by formula If, wherein R₇ represents H or alkyl.

In certain embodiments, the present invention relates to a salt represented by formula I and the attendant definitions, wherein the onium cation is represented by formula If, wherein R₇ represents alkyl.

In certain embodiments, the present invention relates to a salt represented by formula I and the attendant definitions, wherein the onium cation is represented by formula If, wherein Z represents H.

In certain embodiments, the present invention relates to a salt represented by formula I and the attendant definitions, wherein the onium cation is represented by formula If, wherein L represents (C(R₃)₂)ₙ.

In certain embodiments, the present invention relates to a salt represented by formula I and the attendant definitions, wherein the onium cation is represented by formula If, wherein A represents an anion of formula Ia.

In certain embodiments, the present invention relates to a salt represented by formula I and the attendant definitions, wherein the onium cation is represented by formula If, wherein A⁻ represents an anion of formula Ib.

In certain embodiments, the present invention relates to a salt represented by formula I and the attendant definitions, wherein the onium cation is represented by formula If, wherein A⁻ represents an anion of formula Ic.

In certain embodiments, the present invention relates to a salt represented by formula I and the attendant definitions, wherein the onium cation is represented by formula If, wherein M is NR₅ and R₅ represents alkyl or is represented by formula If-a; and Z represents H.

In certain embodiments, the present invention relates to a salt represented by formula I and the attendant definitions, wherein the onium cation is represented by formula If, wherein M is NR₅ and R₅ represents alkyl or is represented by formula If-a; Z represents H; and L represents (C(R₃)₂)ₙ.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; and A⁻ represents an anion of formula Ia.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; and A⁻ represents an anion of formula Ib.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; and A⁻ represents an anion of formula Ic.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; and R₆ represents H or alkyl.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; and A⁻ represents an anion of formula Ia.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; and A⁻ represents an anion of formula Ib.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; and A⁻ represents an anion of formula Ic.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; and R₇ represents H or alkyl.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; R₇ represents H or alkyl; and A⁻ represents an anion of formula Ia.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; R₇ represents H or alkyl; and A⁻ represents an anion of formula Ib.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; R₇ represents H or alkyl; and A⁻ represents an anion of formula Ic.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; and R₇ represents H.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; R₇ represents H; and A⁻ represents an anion of formula Ia, wherein the two R groups taken together represent an aryl ring.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; R₇ represents H; and A⁻ represents an anion of formula Ib, wherein 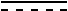
represents a double bond.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; R₇ represents H; and A⁻ represents an anion of formula Ib, wherein 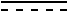
represents a double bond and R represents H.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; R₇ represents H; and A⁻ represents an anion of formula Ib, wherein 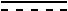
represents a double bond, R represents H, and R₁ represents methyl.

In certain embodiments, the present invention relates to a salt represented by formula I and the attendant definitions, wherein the onium cation is represented by formula If, wherein M is NR₅ and R₅ represents alkyl or is represented by formula If-a; Z represents H; L represents (C(R₃)₂)ₙ; R₆ represents H or alkyl; R₇ represents H; and A⁻ represents an anion of formula Ic, wherein o represents 0.

In certain embodiments, the present invention relates to a salt represented by formula I and the attendant definitions, wherein the onium cation is represented by formula If, wherein M is NR₅ and R₅ represents alkyl; Z represents H; L represents (C(R₃)₂)ₙ; R₆ represents H or alkyl; R₇ represents H; and A⁻ represents an anion of formula Ic, wherein o represents 0, and R₁ represents H.

In certain embodiments, the present invention relates to a salt represented by formula I and the attendant definitions, wherein the onium cation is represented by formula If, wherein M is NR₅ and R₅ represents methyl; Z represents H; L represents —(CH₂)₄—; R₆ represents H; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is NR₅ and R₅ represents methyl; Z represents H; L represents —(CH₂)₅—; R₆ represents H; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is NR₅ and R₅ represents methyl; Z represents H; L represents —(CH₂)₆—; R₆ represents H; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is NR₅ and R₅ represents methyl; Z represents H; L represents —(CH₂)₉—; R₆ represents H; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is NR₅ and R₅ represents n-butyl; Z represents H; L represents —(CH₂)—; R₆ represents H; R₇ represents methyl; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is NR₅ and R₅ represents methyl; Z represents H; L represents —(CH₂)₄—; R₆ represents H or alkyl; R₇ represents H; and A⁻ represents an anion of formula Ib, wherein 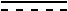
represents a double bond, R represents H, and R₁ represents methyl.

In certain embodiments, the present invention relates to a salt represented by formula I and the attendant definitions, wherein the onium cation is represented by formula If, wherein M is NR₅ and R₅ represents methyl; Z represents H; L represents —(CH₂)₆—; R₆ represents H or alkyl; R₇ represents H; and A⁻ represents an anion of formula Ib, wherein 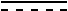
represents a double bond, R represents H, and R₁ represents methyl.

In certain embodiments, the present invention relates to a salt represented by formula I and the attendant definitions, wherein the onium cation is represented by formula If, wherein M is NR₅ and R₅ represents methyl; Z represents H; L represents —(CH₂)₉—; R₆ represents H or alkyl; R₇ represents H; and A⁻ represents an anion of formula Ib, wherein 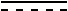
represents a double bond, R represents H, and R₁ represents methyl.

In certain embodiments, the present invention relates to a salt represented by formula I and the attendant definitions, wherein the onium cation is represented by formula If, wherein M is NR₅ and R₅ represents methyl; Z represents H; L represents —(CH₂)₆—; R₆ represents H; R₇ represents H; and A⁻ represents an anion of formula Ic, wherein o represents 0, and R₁ represents H.

In certain embodiments, the present invention relates to a salt represented by formula I and the attendant definitions, wherein the onium cation is represented by formula If, wherein M is NR₅ and R₅ is represented by formula If-a; Z represents H; n is 0; R₆ represents H; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is NR₅ and R₅ is represented by formula If-a, wherein Ar represents a substituted benzene ring; Z represents H; n is 0; R₆ represents H; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is NR₅ and R₅ is represented by formula If-a, wherein Ar represents a substituted benzene ring and W represents 0; Z represents H; n is 0; R₆ represents H; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is NR₅ and R₅ is

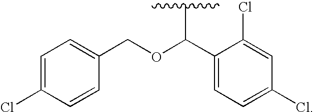

In certain embodiments, the present invention relates to a salt represented by formula I and the attendant definitions, wherein the onium cation is represented by formula If, wherein M is NR₅ and R₅ is 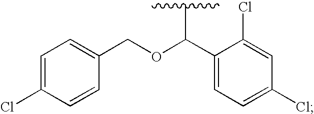
Z represents H; n is O; R₆ represents H; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is NR₅ and R₅ is represented by formula If-a; Z represents H; n is 0; R₆ represents H; R₇ represents H; and A⁻ represents an anion of formula Ib, wherein 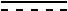
represents a double bond, R represents H, and R₁ represents methyl.

In certain embodiments, the present invention relates to a salt represented by formula I and the attendant definitions, wherein the onium cation is represented by formula If, wherein M is NR₅ and R₅ is represented by formula If-a, wherein Ar represents a substituted benzene ring; Z represents H; n is 0; R₆ represents H; R₇ represents H; and A⁻ represents an anion of formula Ib, wherein 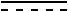
represents a double bond, R represents H, and R₁ represents methyl.

In certain embodiments, the present invention relates to a salt represented by formula I and the attendant definitions, wherein the onium cation is represented by formula If, wherein M is NR₅ and R₅ is represented by formula If-a, wherein Ar represents a substituted benzene ring and W represents 0; Z represents H; n is 0; R₆ represents H; R₇ represents H; and A⁻ represents an anion of formula Ib, wherein 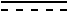
represents a double bond, R represents H, and R₁ represents methyl.

In certain embodiments, the present invention relates to a salt represented by formula I and the attendant definitions, wherein the onium cation is represented by formula If, wherein M is NR₅ and R₅ is 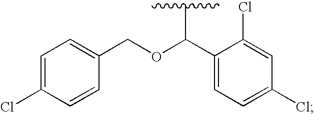
Z represents H; n is O; R₆ represents H; R₇ represents H; and A⁻ represents an anion of formula Ib, wherein 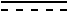
represents a double bond, R represents H, and R₁ represents methyl.

In certain embodiments, the present invention relates to a salt represented by formula I and the attendant definitions, wherein the onium cation is represented by formula If, wherein M is NR₅ and R₅ is represented by formula If-a; Z represents H; n is 0; R₆ represents H; R₇ represents H; and A⁻ represents an anion of formula Ic, wherein o represents 0, and R₁ represents H.

In certain embodiments, the present invention relates to a salt represented by formula I and the attendant definitions, wherein the onium cation is represented by formula If, wherein M is NR₅ and R₅ is represented by formula If-a, wherein Ar represents a substituted benzene ring; Z represents H; n is 0; R₆ represents H; R₇ represents H; and A⁻ represents an anion of formula Ic, wherein o represents 0, and R₁ represents H.

In certain embodiments, the present invention relates to a salt represented by formula I and the attendant definitions, wherein the onium cation is represented by formula If, wherein M is NR₅ and R₅ is represented by formula If-a, wherein Ar represents a substituted benzene ring and W represents 0; Z represents H; n is 0; R₆ represents H; R₇ represents H; and A⁻ represents an anion of formula Ic, wherein o represents 0, and R₁ represents H.

In certain embodiments, the present invention relates to a salt represented by formula I and the attendant definitions, wherein the onium cation is represented by formula If, wherein M is NR₅ and R₅ is 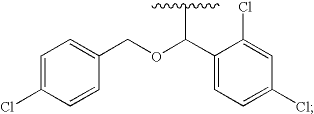
Z represents H; n is O; R₆ represents H; R₇ represents H; and A⁻ represents an anion of formula Ic, wherein o represents 0, and R₁ represents H.

In certain embodiments, the present invention relates to a salt represented by formula I and the attendant definitions, wherein the onium cation is represented by formula If, wherein M is S; and Z represents H.

In certain embodiments, the present invention relates to a salt represented by formula I and the attendant definitions, wherein the onium cation is represented by formula If, wherein M is S; Z represents H; and L represents (C(R₃)₂)ₙ.

In certain embodiments, the present invention relates to a salt represented by formula I and the attendant definitions, wherein the onium cation is represented by formula If, wherein M is S; Z represents H; L represents (C(R₃)₂)ₙ; and A⁻ represents an anion of formula Ia.

In certain embodiments, the present invention relates to a salt represented by formula I and the attendant definitions, wherein the onium cation is represented by formula If, wherein M is S; Z represents H; L represents (C(R₃)₂)ₙ; and A⁻ represents an anion of formula Ib.

In certain embodiments, the present invention relates to a salt represented by formula I and the attendant definitions, wherein the onium cation is represented by formula If, wherein M is S; Z represents H; L represents (C(R₃)₂)ₙ; and A⁻ represents an anion of formula Ic.

In certain embodiments, the present invention relates to a salt represented by formula I and the attendant definitions, wherein the onium cation is represented by formula If, wherein M is S; Z represents H; L represents (C(R₃)₂)ₙ; and one R₆ represents alkyl.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; and A⁻ represents an anion of formula Ia.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; and A⁻ represents an anion of formula Ib.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; and A⁻ represents an anion of formula Ic.

In certain embodiments, the present invention relates to a salt represented by formula I and the attendant definitions, wherein the onium cation is represented by formula If, wherein M S; Z represents H; L represents (C(R₃)₂)ₙ; one R₆ represents alkyl; and R₇ represents H or alkyl.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; R₇ represents H or alkyl; and A⁻ represents an anion of formula Ia.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; R₇ represents H or alkyl; and A⁻ represents an anion of formula Ib.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; R₇ represents H or alkyl; and A⁻ represents an anion of formula Ic.

In certain embodiments, the present invention relates to a salt represented by formula I and the attendant definitions, wherein the onium cation is represented by formula If, wherein M is S; Z represents H; L represents (C(R₃)₂)ᵣ; one R₆ represents alkyl; and R₇ represents H.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; R₇ represents H; and A⁻ represents an anion of formula Ia, wherein the two R groups taken together represent an aryl ring.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; R₇ represents H; and A⁻ represents an anion of formula Ib, wherein 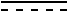
represents a double bond.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; R₇ represents H; and A⁻ represents an anion of formula Ib, wherein 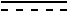
represents a double bond and R represents H.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; R₇ represents H; and A⁻ represents an anion of formula Ib, wherein 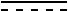
represents a double bond, R represents H, and R₁ represents methyl.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; R₇ represents H; and A⁻ represents an anion of formula Ic, wherein o represents 0.

In certain embodiments, the present invention relates to a salt represented by formula I and the attendant definitions, wherein the onium cation is represented by formula If, wherein M is S; Z represents H; L represents (C(R₃)₂)ₙ; one R₆ represents alkyl; R₇ represents H; and A⁻ represents an anion of formula Ic, wherein o represents 0, and R₁ represents H.

In certain embodiments, the present invention relates to a salt represented by formula I and the attendant definitions, wherein the onium cation is represented by formula If, wherein M is S; Z represents H; L represents —(CH₂)₄—; one R₆ represents methyl; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is S; Z represents H; L represents —(CH₂)₄—; one R₆ represents methyl; R₇ represents H; and A represents an anion of formula Ia, wherein the two R groups taken together represent a fused benzene ring.

In certain embodiments, the present invention relates to a salt represented by formula I and the attendant definitions, wherein the onium cation is represented by formula If, wherein M is S; Z represents H; L represents —(CH₂)₅—; one R₆ represents methyl; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is S; Z represents H; L represents —(CH₂)₆—; one R₆ represents methyl; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is S; Z represents H; L represents —(CH₂)₉—; one R₆ represents methyl; R₇ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula If, wherein M is S; Z represents H; L represents —(CH₂)₄—; one R₆ represents methyl; R₇ represents H; and A⁻ represents an anion of formula Ib, wherein 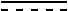
represents a double bond, R represents H, and R₁ represents methyl.

In certain embodiments, the present invention relates to a salt represented by formula I and the attendant definitions, wherein the onium cation is represented by formula If, wherein M is S; Z represents H; L represents —(CH₂)₆—; one R₆ represents methyl; R₇ represents H; and A⁻ represents an anion of formula Ib, wherein 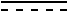
represents a double bond, R represents H, and R₁ represents methyl.

In certain embodiments, the present invention relates to a salt represented by formula I and the attendant definitions, wherein the onium cation is represented by formula If, wherein M is S; Z represents H; L represents —(CH₂)₉—; one R₆ represents methyl; R₇ represents H; and A⁻ represents an anion of formula Ib, wherein 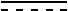
represents a double bond, R represents H, and R₁ represents methyl.

In certain embodiments, the present invention relates to a salt represented by formula I and the attendant definitions, wherein the onium cation is represented by formula If, wherein M is S; Z represents H; L represents —(CH₂)₆—; one R₆ represents methyl; R₇ represents H; and A⁻ represents an anion of formula Ic, wherein o represents 0, and R₁ represents H.

In certain embodiments, the present invention relates to a salt represented by formula I and the attendant definitions, wherein the onium cation is represented by formula Ig: 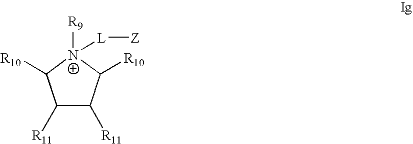
wherein, independently for each occurrence:

R₉ represents alkyl, fluoroalkyl, cycloalkyl, aryl, heteroaryl, aralkyl, heteroaralkyl, or —(CH₂)ₙ—R₈;
R₁₀ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R₁₁ represents H, alkyl, fluoroalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —OC(O)R′—, —CO₂R₉, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₉;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 1-10 inclusive.

In certain embodiments, the present invention relates to a salt represented by formula I and the attendant definitions, wherein the onium cation is represented by formula Ig, wherein R₉ represents alkyl.

In certain embodiments, the present invention relates to a salt represented by formula I and the attendant definitions, wherein the onium cation is represented by formula Ig, wherein R₁₀ represents H or alkyl.

In certain embodiments, the present invention relates to a salt represented by formula I and the attendant definitions, wherein the onium cation is represented by formula Ig, wherein R₁₁ represents H or alkyl.

In certain embodiments, the present invention relates to a salt represented by formula I and the attendant definitions, wherein the onium cation is represented by formula Ig, wherein R₁ represents H.

In certain embodiments, the present invention relates to a salt represented by formula I and the attendant definitions, wherein the onium cation is represented by formula Ig, wherein Z represents H or —OC(O)R′.

In certain embodiments, the present invention relates to a salt represented by formula I and the attendant definitions, wherein the onium cation is represented by formula Ig, wherein L represents (C(R₃)₂)ₙ.

In certain embodiments, the present invention relates to a salt represented by formula I and the attendant definitions, wherein the onium cation is represented by formula Ig, wherein A⁻ represents an anion of formula Ia.

In certain embodiments, the present invention relates to a salt represented by formula I and the attendant definitions, wherein the onium cation is represented by formula Ig, wherein A⁻ represents an anion of formula Ib.

In certain embodiments, the present invention relates to a salt represented by formula I and the attendant definitions, wherein the onium cation is represented by formula Ig, wherein A⁻ represents an anion of formula Ic.

In certain embodiments, the present invention relates to a salt represented by formula I and the attendant definitions, wherein the onium cation is represented by formula Ig, wherein R₉ represents alkyl; and Z represents H or —OC(O)R′.

In certain embodiments, the present invention relates to a salt represented by formula I and the attendant definitions, wherein the onium cation is represented by formula Ig, wherein R₉ represents alkyl; Z represents H or —OC(O)R′; and L represents (C(R₃)₂)ₙ.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; and A⁻ represents an anion of formula Ia.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; and A⁻ represents an anion of formula Ib.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; and A⁻ represents an anion of formula Ic.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; and R₁₀ represents H or alkyl.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; R₁₀ represents H or alkyl; and A⁻ represents an anion of formula Ia.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; R₁₀ represents H or alkyl; and A⁻ represents an anion of formula Ib.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; R₁₀ represents H or alkyl; and A⁻ represents an anion of formula Ic.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; R₁₀ represents H or alkyl; and R₁₁ represents H or alkyl.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; R₁₀ represents H or alkyl; R₁ represents H or alkyl; and A⁻ represents an anion of formula Ia.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; R₁₀ represents H or alkyl; R₁₁ represents H or alkyl; and A⁻ represents an anion of formula Ib.

In certain embodiments, the present invention relates to a salt represented by formula I and the attendant definitions, wherein the onium cation is represented by formula Ig, wherein R₉ represents alkyl; Z represents H or —OC(O)R′; L represents (C(R₃)₂)ₙ; R₁₀ represents H or alkyl; R₁ represents H or alkyl; and A⁻ represents an anion of formula Ic.

In certain embodiments, the present invention relates to a salt represented by formula I and the attendant definitions, wherein the onium cation is represented by formula Ig, wherein R₉ represents alkyl; Z represents —OC(O)R′; L represents (C(R₃)₂)ₙ; R₁₀ represents H; and R₁ represents H.

In certain embodiments, the present invention relates to a salt represented by formula I and the attendant definitions, wherein the onium cation is represented by formula Ig, wherein R₉ represents alkyl; Z represents —OC(O)R′; L represents (C(R₃)₂)ₙ; R₁₀ represents H; R₁₁, represents H; and A⁻ represents an anion of formula Ia, wherein the two R groups taken together represent an aryl ring.

In certain embodiments, the present invention relates to a salt represented by formula I and the attendant definitions, wherein the onium cation is represented by formula Ig, wherein R₉ represents alkyl; Z represents —OC(O)R′; L represents (C(R₃)₂)ₙ; R₁₀ represents H; R₁₁ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula Ig, wherein R₉ represents alkyl; Z represents —OC(O)R′; L represents (C(R₃)₂)ₙ; R₁₀ represents H; R₁₁ represents H; and A⁻ represents an anion of formula Ib, wherein 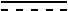
represents a double bond.

In certain embodiments, the present invention relates to a salt represented by formula I and the attendant definitions, wherein the onium cation is represented by formula Ig, wherein R₉ represents alkyl; Z represents —OC(O)R′; L represents (C(R₃)₂)ₙ; R₁₀ represents H; R₁₁ represents H; and A⁻ represents an anion of formula Ib, wherein 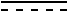
represents a double bond and R represents H.

In certain embodiments, the present invention relates to a salt represented by formula I and the attendant definitions, wherein the onium cation is represented by formula Ig, wherein R₉ represents alkyl; Z represents —OC(O)R′; L represents (C(R₃)₂)ₙ; R₁₀ represents H; R₁₁ represents H; and A⁻ represents an anion of formula Ib, wherein 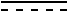
represents a double bond, R represents H, and R₁ represents methyl.

In certain embodiments, the present invention relates to a salt represented by formula I and the attendant definitions, wherein the onium cation is represented by formula Ig, wherein R₉ represents alkyl; Z represents —OC(O)R′; L represents (C(R₃)₂)ₙ; R₁₀ represents H; R₁₁ represents H; and A⁻ represents an anion of formula Ic, wherein o represents 0.

In certain embodiments, the present invention relates to a salt represented by formula I and the attendant definitions, wherein the onium cation is represented by formula Ig, wherein R₉ represents alkyl; Z represents —OC(O)R′; L represents (C(R₃)₂)ₙ; R₁₀ represents H; R₁₁ represents H; and A⁻ represents an anion of formula Ic, wherein o represents 0, and R₁ represents H.

In certain embodiments, the present invention relates to a salt represented by formula I and the attendant definitions, wherein the onium cation is represented by formula Ig, wherein R₉ represents methyl; Z represents —OC(O)CH₂CH₂CH₃; L represents —(CH₂)₂—; R₁₀ represents H; R₁₁ represents H; and A⁻ represents an anion of formula Ia, wherein the two R groups taken together represent a fused benzene ring.

In certain embodiments, the present invention relates to a salt represented by formula I and the attendant definitions, wherein the onium cation is represented by formula Ih: 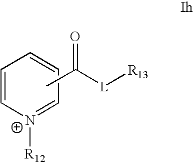
wherein:

L represents O, NR₁₂, or S;
R₁₂ represents H, alkyl, fluoroalkyl, aryl, heteroaryl, aralkyl, heteroaralkyl, or —(CH₂)ₙ—R₈;
R₁₃ represents H, alkyl, fluoroalkyl, aryl, heteroaryl, aralkyl, heteroaralkyl, or —(CH₂)ₙ—R₈; and R₈ represents cycloalkyl, aryl, or heteroaryl.

In certain embodiments, the present invention relates to a salt represented by formula I and the attendant definitions, wherein the onium cation is represented by formula Ih, wherein R₁₂ represents alkyl.

In certain embodiments, the present invention relates to a salt represented by formula I and the attendant definitions, wherein the onium cation is represented by formula Ih, wherein L represents O.

In certain embodiments, the present invention relates to a salt represented by formula I and the attendant definitions, wherein the onium cation is represented by formula Ih, wherein R₁₃ represents alkyl.

In certain embodiments, the present invention relates to a salt represented by formula I and the attendant definitions, wherein the onium cation is represented by formula Ih, wherein A⁻ represents an anion of formula Ia.

In certain embodiments, the present invention relates to a salt represented by formula I and the attendant definitions, wherein the onium cation is represented by formula Ih, wherein A⁻ represents an anion of formula Ib.

In certain embodiments, the present invention relates to a salt represented by formula I and the attendant definitions, wherein the onium cation is represented by formula Ih, wherein A⁻ represents an anion of formula Ic.

In certain embodiments, the present invention relates to a salt represented by formula I and the attendant definitions, wherein the onium cation is represented by formula Ih, wherein R₁₂ represents alkyl; and L represents O.

In certain embodiments, the present invention relates to a salt represented by formula I and the attendant definitions, wherein the onium cation is represented by formula Ih, wherein R₁₂ represents alkyl; L represents O; and R₁₃ represents alkyl.

In certain embodiments, the present invention relates to a salt represented by formula I and the attendant definitions, wherein the onium cation is represented by formula Ih, wherein R₁₂ represents alkyl; L represents O; R₁₃ represents alkyl; and A⁻ represents an anion of formula Ia.

In certain embodiments, the present invention relates to a salt represented by formula I and the attendant definitions, wherein the onium cation is represented by formula Ih, wherein R₁₂ represents alkyl; L represents O; R₁₃ represents alkyl; and A⁻ represents an anion of formula Ib.

In certain embodiments, the present invention relates to a salt represented by formula I and the attendant definitions, wherein the onium cation is represented by formula Ih, wherein R₁₂ represents alkyl; L represents O; R₁₃ represents alkyl; and A⁻ represents an anion of formula Ic.

In certain embodiments, the present invention relates to a salt represented by formula I and the attendant definitions, wherein the onium cation is represented by formula Ih, wherein R₁₂ represents alkyl; L represents O; R₁₃ represents alkyl; and A⁻ represents an anion of formula Ia, wherein the two R groups taken together represent an aryl ring.

In certain embodiments, the present invention relates to a salt represented by formula I and the attendant definitions, wherein the onium cation is represented by formula Ii, wherein R₁₂ represents alkyl; L represents O; R₁₃ represents alkyl; and A⁻ represents an anion of formula Ia, wherein the two R groups taken together represent a fused benzene ring.

In certain embodiments, the present invention relates to a salt represented by formula I and the attendant definitions, wherein the onium cation is represented by formula Ih, wherein R₁₂ represents alkyl; L represents O; R₁₃ represents alkyl; and A⁻ represents an anion of formula Ib, wherein 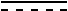
represents a double bond.

In certain embodiments, the present invention relates to a salt represented by formula I and the attendant definitions, wherein the onium cation is represented by formula Ih, wherein R₁₂ represents alkyl; L represents O; R₁₃ represents alkyl; and A⁻ represents an anion of formula Ib, wherein 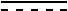
represents a double bond and R represents H.

In certain embodiments, the present invention relates to a salt represented by formula I and the attendant definitions, wherein the onium cation is represented by formula Ih, wherein R₁₂ represents alkyl; L represents O; R₁₃ represents alkyl; and A⁻ represents an anion of formula Ib, wherein 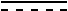
represents a double bond, R represents H, and R₁ represents methyl.

In certain embodiments, the present invention relates to a salt represented by formula I and the attendant definitions, wherein the onium cation is represented by formula Ih, wherein R₁₂ represents alkyl; L represents O; R₁₃ represents alkyl; and A⁻ represents an anion of formula Ic, wherein o represents 0.

In certain embodiments, the present invention relates to a salt represented by formula I and the attendant definitions, wherein the onium cation is represented by formula Ih, wherein R₁₂ represents alkyl; L represents O; R₁₃ represents alkyl; and A⁻ represents an anion of formula Ic, wherein o represents 0, and R₁ represents H.

In certain embodiments, the present invention relates to a salt represented by formula I and the attendant definitions, wherein the onium cation is represented by formula Ih, wherein R₁₂ represents methyl; L represents O; R₁₃ represents n-butyl; and A⁻ represents an anion of formula Ia, wherein the two R groups taken together represent a fused benzene ring.

In certain embodiments, the present invention relates to a salt represented by formula I and the attendant definitions, wherein the onium cation is represented by formula Ih, wherein R₁₂ represents methyl; L represents O; R₁₃ represents n-butyl; and A⁻ represents an anion of formula Ib, wherein 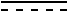
represents a double bond, R represents H, and R₁ represents methyl.

In certain embodiments, the present invention relates to a salt represented by formula I and the attendant definitions, wherein the onium cation is represented by formula Ih, wherein R₁₂ represents methyl; L represents O; R₁₃ represents n-butyl; and A⁻ represents an anion of formula Ic, wherein o represents 0, and R₁ represents H.

Methods of the Invention

In certain embodiments, the present invention relates to the use of an IL with an appended amine (e.g., primary, secondary, tertiary, or heterocyclic) for the capture from the gas phase of an acidic gas, including but not limited to H₂S, CO₂, COS, SO₂, and SO₃.

In certain embodiments, the present invention relates to the use of an IL with an appended amine (e.g., primary, secondary, tertiary, or heterocyclic) in conjunction with water for the capture of an acidic gas from the gas phase.

In certain embodiments, the present invention relates to the use of an IL with an appended amine (e.g., primary, secondary, tertiary, or heterocyclic) dissolved in a molecular solvent or other ionic liquid for the capture of an acidic gas from the gas phase.

In certain embodiments, the present invention relates to the use of an IL with an appended amine dissolved in water or other solvent as a non-odorous, non-volatile base for a general-base-catalyzed reaction.

In certain embodiments, the present invention relates to the use of an IL with an appended amine as a scavaging agent for an amine-reactive material in the solution phase.

In certain embodiments, the present invention relates to the use of an IL with an appended amine as a solvent.

In certain embodiments, the present invention relates to the use of an IL with an appended amine (e.g., primary, secondary, tertiary or heterocyclic) alone or in conjunction with an organic molecule, such as salicylaldehyde, for the extraction of a metal ion from an aqueous solution.

In certain embodiments, the present invention relates to the use of an IL with an appended amine in conjunction with an ion-exchange resin, clay or zeolite for any of the aforementioned applications.

In certain embodiments, the present invention relates to the use of an IL with an appended acidic group for general or specific acid catalysis, either as a pure material, or as a solution in another ionic liquid or molecular solvent. Such reactions include, but are not limited to, Fischer esterification, pinnacol rearrangement, alcohol dehydration, rearrangements, isomerizations, Friedel-Crafts alkylation and acylation, or aromatic nitration.

In certain embodiments, the present invention relates to the use of an IL with an appended acidic group as a scavaging agent for an acid-reactive material in the gas or solution phase.

In certain embodiments, the present invention relates to the use of an IL with an appended acidic group as a dehydrating or drying agent.

In certain embodiments, the present invention relates to the use of an IL with an appended acid in conjunction with an ion-exchange resin, clay or zeolite for any of the aforementioned applications.

In certain embodiments, the present invention relates to the use of an IL with an appended acidic group as a solvent.

In certain embodiments, the present invention relates to the use of an IL with an appended fluoroketone or fluoroalcohol group as a solvent; as an acid; or as an activator of peroxide for use in an oxidation reaction.

In certain embodiments, the present invention relates to the use of an IL with an appended sulfone, sulfoxide or sulfonamide group in a liquid-liquid or liquid-gas separation, including a separation in the refining of petroleum or petrochemicals.

In certain embodiments, the present invention relates to the use of an IL with an appended sulfone, sulfoxide or sulfonamide group as a solvent for a polar molecule, including but not limited to biomolecules, such as saccharides, amino acids, nucleic acids, proteins, enzymes, DNA and RNA.

In certain embodiments, the present invention relates to the use of an IL with an appended sulfone, sulfoxide or sulfonamide group as a solvent.

In certain embodiments, the present invention relates to the use of an IL with an appended sulfone, sulfoxide or sulfonamide group as a phase-transfer adjuvant for use in conjunction with a supercritical solvent, e.g., supercritical CO₂.

In certain embodiments, the present invention relates to the use of an IL with an appended sulfonyl halide group as a scavaging reagent for use in conjunction with a reactive species.

In certain embodiments, the present invention relates to the use of an IL with an appended sulfone or sulfoxide group in conjunction with ion exchangeable materials, such as ion exchange resins, clays, and zeolites, for any of the aforementioned uses.

In certain embodiments, the present invention relates to the use of an IL with an appended amide, urea or thiourea group in a liquid-liquid or liquid-gas separation, including separations in the refining of petroleum or petrochemicals.

In certain embodiments, the present invention relates to the use of an IL with an appended amide, urea or thiourea group as a solvent for a polar molecule, including but not limited to biomolecules, such as saccharides, amino acids, nucleic acids, proteins, enzymes, DNA and RNA.

In certain embodiments, the present invention relates to the use of an IL with an appended amide, urea or thiourea group as a solvent.

In certain embodiments, the present invention relates to the use of an IL with an appended amide, urea or thiourea group in conjunction with an ion exchangeable material, such as ion exchange resins, clays, and zeolites, for any of the aforementioned uses.

In certain embodiments, the present invention relates to the use of an IL with an appended amide, urea or thiourea group as a phase-transfer adjuvant for use in conjunction with a supercritical solvent, e.g., supercritical CO₂.

In certain embodiments, the present invention relates to the use of a phosphoramide appended IL, alone or in conjunction with another ionic liquid or a molecular solvent, as a solvent or for the extraction of a metal from an ore or immiscible solution phase.

In certain embodiments, the present invention relates to the use of a functionalized IL as a solvent, reagent-solvent, or a catalyst-solvent for a polymerization or a polymer-processing operation.

In certain embodiments, the present invention relates to the use of a functionalized IL as an anti-static agent, e.g., in a solution, petroleum or a petrochemical.

In certain embodiments, the present invention relates to a method of removing carbon dioxide, carbonyl sulfide, sulfur dioxide, sulfur trioxide, hydrogen sulfide or a carbonyl-containing compound from a gaseous or liquid mixture, comprising the step of exposing a gaseous or liquid mixture to a salt represented by formula I:
C⁺A⁻ I
wherein: C⁺ represents an onium cation, and A⁻ represents an anion of formula Ia: 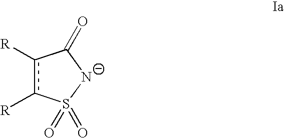
wherein, independently for each occurrence:

R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈; or the two R taken together represent a substituted or unsubstituted fused cycloalkyl, cycloalkenyl, aryl, or heteroaryl ring;
R₈ represents cycloalkyl, aryl, or heteroaryl;
n represents an integer from 1-10 inclusive; and 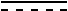
represents a single or double bond; or
an anion of formula Ib: 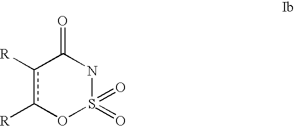
wherein:
R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
R₁ represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
or R and R₁ taken together represent a substituted or unsubstituted fused cycloalkyl, cycloalkenyl, aryl, or heteroaryl ring;
R₈ represents cycloalkyl, aryl, or heteroaryl;
n represents an integer from 1-10 inclusive; and 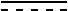
represents a single or double bond; or
an anion of formula Ic: 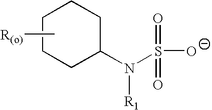
Ic
wherein, independently for each occurrence:
R is halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈, or any two adjacent R taken together represent a substituted or unsubstituted fused cycloalkyl, cycloalkenyl, aryl, or heteroaryl ring;
R₁ is H, halide, alkyl, alkenyl, alkynyl, cycloalky, aryl, heteroaryl, aralkyl, heteroaralkyl, hydroxy, amino, alkoxy, carboxyl, keto, aldehyde, or —(CH₂)ₙ—R₈;
R₈ represents cycloalkyl, aryl, or heteroaryl;
n represents an integer from 1-10 inclusive; and
o represents an integer from 0 to 11 inclusive; and wherein the onium cation has one of the following formulas: 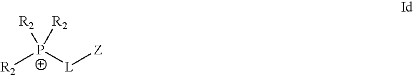
   wherein, independently for each occurrence:
R₂ represents alkyl, fluoroalkyl, cycloalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —CO₂R₂, —C(O)N(R″)₂, —C(O)N(R″)N(R″)₂, —N(R′)₂, —OR′, —SR′, —S(O)R″, —S(O)₂R″, —CN, —N(R″)P(O)(R)₂, —C(OR′)(R″)₂, alkenyl, or alkynyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
Ar represents aryl or heteroaryl;
J represents O, S, NR′, cycloalkyl, or heterocyclyl;
R₈ represents cycloalkyl, aryl, or heteroaryl;
m represents an integer from 1-10 inclusive; and
n represents an integer in the range 1-10 inclusive; or 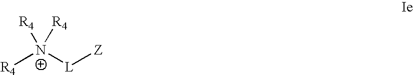
   wherein, independently for each occurrence:
R₄ represents alkyl, fluoroalkyl, cycloalkyl, aryl, heteroaryl, aralkyl, heteroaralkyl, or —(CH₂)ₙ—R₈; or ⁺N(R₄)₃ taken together represents pyridinium, imidazolium, benzimidazolium, pyrazolium, benzpyrazolium, indazolium, thiazolium, benzthiazolium, oxazolium, benzoxazolium, isoxazolium, isothiazolium, imdazolidenium, guanidinium, quinuclidinium, triazolium, tetrazolium, quinolinium, isoquinolinium, piperidinium, pyrrolidinium, morpholinium, pyridazinium, pyrazinium, piperaziniurn, triazinium, azepinium, or diazepinium;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —OC(O)R′, —CO₂R₄, —C(O)N(R″)₂, —C(O)N(R″)N(R″)₂, —N(R′)₂, —OR′, —SR′, —S(O)R″, —S(O)₂R″, —CN, —N(R″)P(O)(R₄)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 1-10 inclusive; or 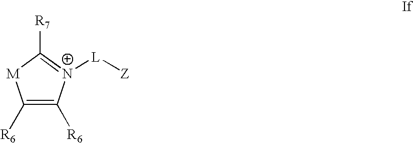
   wherein, independently for each occurrence:
M represents NR₅ or S;
R₅ represents H, alkyl, fluoroalkyl, cycloalkyl, aryl, heteroaryl, aralkyl, heteroaralkyl, or —(CH₂)ₙ—R₈; or R₅ is represented by formula If-a: 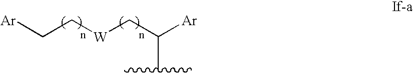
   wherein, independently for each occurrence:
   Ar represents a substituted or unsubstituted aryl or heteroaryl ring; and
   W represents O, NR₇, or S;
R₆ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R₇ represents H, alkyl, fluoroalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —CO₂R₅, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 0-10 inclusive; or 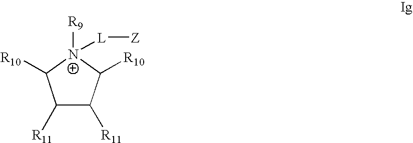
   wherein, independently for each occurrence:

R₉ represents alkyl, fluoroalkyl, cycloalkyl, aryl, heteroaryl, aralkyl, heteroaralkyl, or —(CH₂)ₙ—R₈;
R₁₀ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R₁₁ represents H, alkyl, fluoroalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —OC(O)R′—, —CO₂R₉, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 1-10 inclusive; or 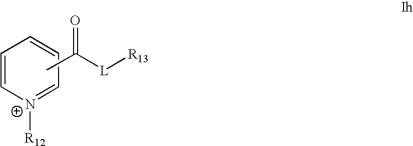
wherein:
L represents O, NR₁₂, or S;
R₁₂ represents H, alkyl, fluoroalkyl, aryl, heteroaryl, aralkyl, heteroaralkyl, or —(CH₂)ₙ—R₈;
R₁₃ represents H, alkyl, fluoroalkyl, aryl, heteroaryl, aralkyl, heteroaralkyl, or —(CH₂)ₙ—R₈; and
R₈ represents cycloalkyl, aryl, or heteroaryl.

In certain embodiments, the present invention relates to the aforementioned method and the attendant definitions, wherein Z represents independently for each occurrence —N(R′)₂.

In certain embodiments, the present invention relates to the aforementioned method and the attendant definitions, wherein said gaseous or liquid mixture is natural gas.

In certain embodiments, the present invention relates to the aforementioned method and the attendant definitions, wherein carbon dioxide is removed.

In certain embodiments, the present invention relates to the aforementioned method and the attendant definitions, wherein the salt is dissolved in water.

In certain embodiments, the present invention relates to the aforementioned method and the attendant definitions, wherein Z represents independently for each occurrence —N(R′)₂; and said gaseous or liquid mixture is natural gas.

In certain embodiments, the present invention relates to a method of transporting carbon dioxide, carbonyl sulfide, sulfur dioxide, sulfur trioxide, hydrogen sulfide or a carbonyl-containing compound from a first gaseous or liquid mixture to a second gaseous or liquid mixture, comprising the steps of exposing a first gaseous or liquid mixture to a salt represented by formula I; and subsequently exposing the salt to a second gaseous or liquid mixture, thereby transporting carbon dioxide, carbonyl sulfide, sulfur dioxide, sulfur trioxide, hydrogen sulfide or a carbonyl-containing compound to the second gaseous or liquid mixture:
C⁺A⁻ I
wherein:
C⁺ represents an onium cation, and
A⁻ represents an anion of formula Ia: 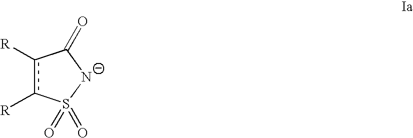
   wherein, independently for each occurrence:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈; or the two R taken together represent a substituted or unsubstituted fused cycloalkyl, cycloalkenyl, aryl, or heteroaryl ring;
   R₈ represents cycloalkyl, aryl, or heteroaryl;
   n represents an integer from 1-10 inclusive; and 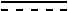
   represents a single or double bond; or
   an anion of formula Ib: 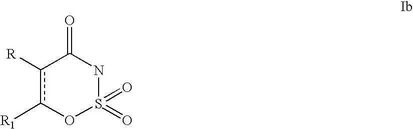
   wherein:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
   R₁ represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
   or R and R₁ taken together represent a substituted or unsubstituted fused cycloalkyl, cycloalkenyl, aryl, or heteroaryl ring;
   R₉ represents cycloalkyl, aryl, or heteroaryl;
   n represents an integer from 1-10 inclusive; and 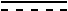
   represents a single or double bond; or
   an anion of formula Ic: 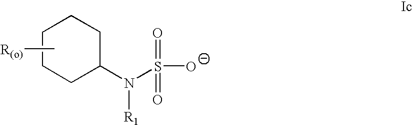
   wherein, independently for each occurrence:
   R is halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈, or any two adjacent R taken together represent a substituted or unsubstituted fused cycloalkyl, cycloalkenyl, aryl, or heteroaryl ring;
   R₁ is H, halide, alkyl, alkenyl, alkynyl, cycloalky, aryl, heteroaryl, aralkyl, heteroaralkyl, hydroxy, amino, alkoxy, carboxyl, keto, aldehyde, or —(CH₂)ₙ—R₈;
   R₈ represents cycloalkyl, aryl, or heteroaryl;
   n represents an integer from 1-10 inclusive; and
   o represents an integer from 0 to 11 inclusive; and
   wherein the onium cation has one of the following formulas: 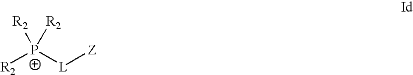
      wherein, independently for each occurrence:
   R₂ represents alkyl, fluoroalkyl, cycloalkyl, aryl, heteroaryl, aralkyl, heteroaralkyl, or —(CH₂)ₙ—R₈;
   L represents (C(R₃)₂)ₙ, (C(R₃)₂)ₙJ(C(R₃)₂)ₘ, or (C(R₃)₂)ₙAr(C(R₃)₂)ₘ;
   Z represents H, —CO₂H, —CO₂R₂, —C(O)N(R″)₂, —C(O)N(R″)N(R″)₂, —N(R′)₂, —OR′, —SR′, —S(O)R″, —S(O)₂R″, —CN, —N(R″)P(O)(R)₂, —C(OR′)(R″)₂, alkenyl, or alkynyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   Ar represents aryl or heteroaryl;
   J represents O, S, NR′, cycloalkyl, or heterocyclyl;
   R₈ represents cycloalkyl, aryl, or heteroaryl;
   m represents an integer from 1-10 inclusive; and
   n represents an integer in the range 1-10 inclusive; or 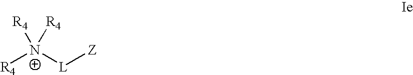
      wherein, independently for each occurrence:
   R₄ represents alkyl, fluoroalkyl, cycloalkyl, aryl, heteroaryl, aralkyl, heteroaralkyl, or —(CH₂)ₙ—R₉; or ⁺N(R₄)₃ taken together represents pyridinium, imidazolium, benzimidazolium, pyrazolium, benzpyrazolium, indazolium, thiazolium, benzthiazolium, oxazolium, benzoxazolium, isoxazolium, isothiazolium, imdazolidenium, guanidinium, quinuclidinium, triazolium, tetrazolium, quinolinium, isoquinolinium, piperidinium, pyrrolidinium, morpholinium, pyridazinium, pyrazinium, piperazinium, triazinium, azepinium, or diazepinium;
   L represents (C(R₃)₂)ₙ, (C(R₃)₂)ₙJ(C(R₃)₂)ₘ, or (C(R₃)₂)ₙAr(C(R₃)₂)ₘ;
   Z represents H, —CO₂H, —OC(O)R′, —CO₂R₄, —C(O)N(R″)₂, —C(O)N(R″)N(R″)₂, —N(R′)₂, —OR′, —SR′, —S(O)R″, —S(O)₂R″, —CN, —N(R″)P(O)(R₄)₂, —C(OR′)(R″)₂, alkenyl, or alkynyl;
   Ar represents aryl or heteroaryl;
   J represents O, S, NR′, cycloalkyl, or heterocyclyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   R₈ represents cycloalkyl, aryl, or heteroaryl;
   m represents an integer from 1-10 inclusive; and
   n represents an integer from 1-10 inclusive; or 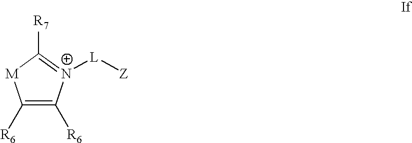
      wherein, independently for each occurrence:
   M represents NR₅ or S;
   R₅ represents H, alkyl, fluoroalkyl, cycloalkyl, aryl, heteroaryl, aralkyl, heteroaralkyl, or —(CH₂)ₙ—R₈; or R₅ is represented by formula If-a: 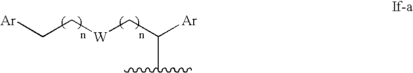
      wherein, independently for each occurrence:
      Ar represents a substituted or unsubstituted aryl or heteroaryl ring; and
      W represents O, NR₇, or S;
   R₆ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R₇ represents H, alkyl, fluoroalkyl, aryl, heteroaryl, aralkyl, heteroaralkyl, or —(CH₂)ₙ—R₈;
   L represents (C(R₃)₂)ₙ, (C(R₃)₂)ₙJ(C(R₃)₂)ₘ, or (C(R₃)₂)ₙAr(C(R₃)₂)ₘ;
   Z represents H, —CO₂H, —CO₂R₅, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
   Ar represents aryl or heteroaryl;
   J represents O, S, NR′, cycloalkyl, or heterocyclyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ᵣ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   R₈ represents cycloalkyl, aryl, or heteroaryl;
   m represents an integer from 1-10 inclusive; and
   n represents an integer from 0-10 inclusive; or 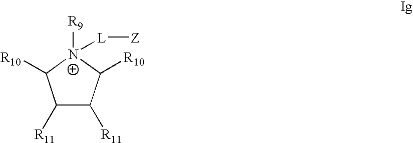
      wherein, independently for each occurrence:
   R₉ represents alkyl, fluoroalkyl, cycloalkyl, aryl, heteroaryl, aralkyl, heteroaralkyl, or —(CH₂)ₙ—R₈;
   R₁₀ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R₁₁, represents H, alkyl, fluoroalkyl, aryl, heteroaryl, aralkyl, heteroaralkyl, or —(CH₂)ₙ—R₈;
   L represents (C(R₃)₂)ₙ, (C(R₃)₂)ₙJ(C(R₃)₂)ₘ, or (C(R₃)₂)ₙAr(C(R₃)₂)ₘ;
   Z represents H, —CO₂H, —OC(O)R′—, —CO₂R₉, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
   Ar represents aryl or heteroaryl;
   J represents O, S, NR′, cycloalkyl, or heterocyclyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   R₈ represents cycloalkyl, aryl, or heteroaryl;
   m represents an integer from 1-10 inclusive; and
   n represents an integer from 1-10 inclusive; or 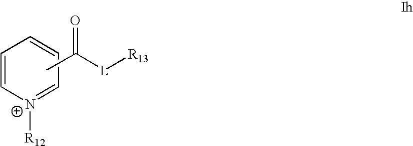
   wherein:

L represents O, NR₁₂, or S;
R₁₂ represents H, alkyl, fluoroalkyl, aryl, heteroaryl, aralkyl, heteroaralkyl, or —(CH₂)ₙ—R₈;
R₁₃ represents H, alkyl, fluoroalkyl, aryl, heteroaryl, aralkyl, heteroaralkyl, or —(CH₂)ₙ—R₈; and
R₈ represents cycloalkyl, aryl, or heteroaryl.

In certain embodiments, the present invention relates to the aforementioned method and the attendant definitions, wherein Z represents independently for each occurrence —N(R′)₂.

In certain embodiments, the present invention relates to the aforementioned method and the attendant definitions, wherein said salt is contained within a semi-permeable membrane.

In certain embodiments, the present invention relates to the aforementioned method and the attendant definitions, wherein Z represents independently for each occurrence —N(R′)₂; and said salt is contained within a semi-permeable membrane.

In certain embodiments, the present invention relates to a method of removing an alkene, alkyne or carbon monoxide from a mixture, comprising the step of exposing a mixture to a complex formed from a transition metal and a salt represented by formula I:
C⁺A⁻ I
wherein:
C⁺ represents an onium cation, and
A⁻ represents an anion of formula Ia: 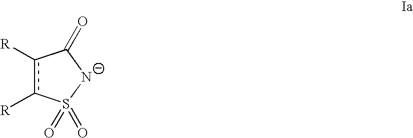
   wherein, independently for each occurrence:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈; or the two R taken together represent a substituted or unsubstituted fused cycloalkyl, cycloalkenyl, aryl, or heteroaryl ring;
   R₅ represents cycloalkyl, aryl, or heteroaryl;
   n represents an integer from 1-10 inclusive; and 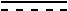
   represents a single or double bond; or
   an anion of formula Ib: 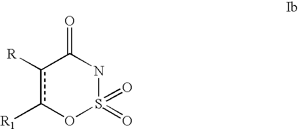
   wherein:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
   R₁ represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
   or R and R₁ taken together represent a substituted or unsubstituted fused cycloalkyl, cycloalkenyl, aryl, or heteroaryl ring;
   R₈ represents cycloalkyl, aryl, or heteroaryl;
   n represents an integer from 1-10 inclusive; and 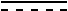
   represents a single or double bond; or
   an anion of formula Ic: 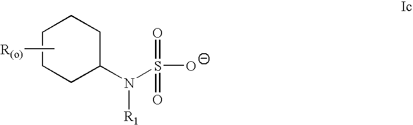
   wherein, independently for each occurrence:
   R is halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈, or any two adjacent R taken together represent a substituted or unsubstituted fused cycloalkyl, cycloalkenyl, aryl, or heteroaryl ring;
   R₁ is H, halide, alkyl, alkenyl, alkynyl, cycloalky, aryl, heteroaryl, aralkyl, heteroaralkyl, hydroxy, amino, alkoxy, carboxyl, keto, aldehyde, or —(CH₂)ₙ—R₈;
   R₈ represents cycloalkyl, aryl, or heteroaryl;
   n represents an integer from 1-10 inclusive; and
   o represents an integer from 0 to 11 inclusive; and wherein the onium cation has one of the following formulas: 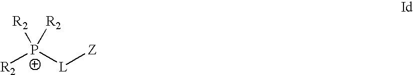
      wherein, independently for each occurrence:
   R₂ represents alkyl, fluoroalkyl, cycloalkyl, aryl, heteroaryl, aralkyl, heteroaralkyl, or —(CH₂)ₙ—R₈;
   L represents (C(R₃)₂)ₙ, (C(R₃)₂)ₙJ(C(R₃)₂)ₘ, or (C(R₃)₂)ₙAr(C(R₃)₂)ₘ;
   Z represents H, —CO₂H, —CO₂R₂, —C(O)N(R″)₂, —C(O)N(R″)N(R″)₂, —N(R′)₂, —OR′, —SR′, —S(O)R″, —S(O)₂R″, —CN, —N(R″)P(O)(R)₂, —C(OR′)(R″)₂, alkenyl, or alkynyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   Ar represents aryl or heteroaryl;
   J represents O, S, NR′, cycloalkyl, or heterocyclyl;
   R₈ represents cycloalkyl, aryl, or heteroaryl;
   m represents an integer from 1-10 inclusive; and
   n represents an integer in the range 1-10 inclusive; or 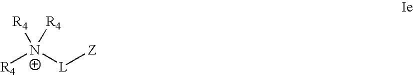
      wherein, independently for each occurrence:
   R₄ represents alkyl, fluoroalkyl, cycloalkyl, aryl, heteroaryl, aralkyl, heteroaralkyl, or —(CH₂)ₙ—R₈; or ⁺N(R₄)₃ taken together represents pyridinium, imidazolium, benzimidazolium, pyrazolium, benzpyrazolium, indazolium, thiazolium, benzthiazolium, oxazolium, benzoxazolium, isoxazolium, isothiazolium, imdazolidenium, guanidinium, quinuclidinium, triazolium, tetrazolium, quinolinium, isoquinolinium, piperidinium, pyrrolidinium, morpholinium, pyridazinium, pyrazinium, piperazinium, triazinium, azepinium, or diazepinium;
   L represents (C(R₃)₂)ₙ, (C(R₃)₂)ₙJ(C(R₃)₂)ₘ, or (C(R₃)₂)ₙAr(C(R₃)₂)ₘ;
   Z represents H, —CO₂H, —OC(O)R′, —CO₂R₄, —C(O)N(R″)₂, —C(O)N(R)N(R″)₂, —N(R′)₂, —OR′, —SR′, —S(O)R″, —S(O)₂R″, —CN, —N(R″)P(O)(R₄)₂, —C(OR′)(R″)₂, alkenyl, or alkynyl;
   Ar represents aryl or heteroaryl;
   J represents O, S, NR′, cycloalkyl, or heterocyclyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   R₈ represents cycloalkyl, aryl, or heteroaryl;
   m represents an integer from 1-10 inclusive; and
   n represents an integer from 1-10 inclusive; or 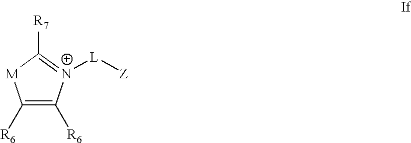
      wherein, independently for each occurrence:
   M represents NR₅ or S;
   R₅ represents H, alkyl, fluoroalkyl, cycloalkyl, aryl, heteroaryl, aralkyl, heteroaralkyl, or —(CH₂)ₙ—R₈; or R₅ is represented by formula If-a: 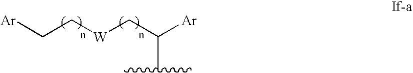
      wherein, independently for each occurrence:
      Ar represents a substituted or unsubstituted aryl or heteroaryl ring; and
      W represents O, NR₇, or S;
   R₆ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R₇ represents H, alkyl, fluoroalkyl, aryl, heteroaryl, aralkyl, heteroaralkyl, or —(CH₂)ₙ—R₈;
   L represents (C(R₃)₂)ₙ, (C(R₃)₂)ₙJ(C(R₃)₂)ₘ, or (C(R₃)₂)ₙAr(C(R₃)₂)ₙ;
   Z represents H, —CO₂H, —CO₂R₅, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
   Ar represents aryl or heteroaryl;
   J represents O, S, NR′, cycloalkyl, or heterocyclyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   R₈ represents cycloalkyl, aryl, or heteroaryl;
   m represents an integer from 1-10 inclusive; and
   n represents an integer from 0-10 inclusive; or 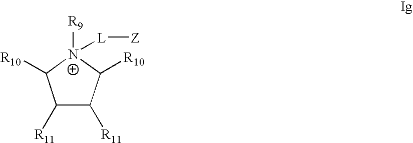
      wherein, independently for each occurrence:
   R₉ represents alkyl, fluoroalkyl, cycloalkyl, aryl, heteroaryl, aralkyl, heteroaralkyl, or —(CH₂)ₙ—R₈;
   R₁₀ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R₁₁ represents H, alkyl, fluoroalkyl, aryl, heteroaryl, aralkyl, heteroaralkyl, or —(CH₂)ₙ—R₈;
   L represents (C(R₃)₂)ₙ, (C(R₃)₂)ₙJ(C(R₃)₂)ₘ, or (C(R₃)₂)ₙAr(C(R₃)₂)ₙ;
   Z represents H, —CO₂H, —OC(O)R′—, —CO₂R₉, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
   Ar represents aryl or heteroaryl;
   J represents O, S, NR′, cycloalkyl, or heterocyclyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   R₈ represents cycloalkyl, aryl, or heteroaryl;
   m represents an integer from 1-10 inclusive; and
   n represents an integer from 1-10 inclusive; or 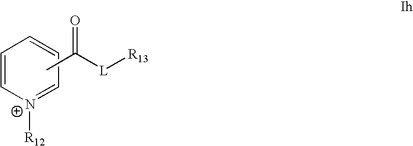
   wherein:
   L represents O, NR₁₂, or S;
   R₁₂ represents H, alkyl, fluoroalkyl, aryl, heteroaryl, aralkyl, heteroaralkyl, or —(CH₂)ₙ—R₈;
   R₁₃ represents H, alkyl, fluoroalkyl, aryl, heteroaryl, aralkyl, heteroaralkyl, or —(CH₂)ₙ—R₈; and
   R₈ represents cycloalkyl, aryl, or heteroaryl.

In certain embodiments, the present invention relates to the aforementioned method and the attendant definitions, wherein Z represents independently for each occurrence alkenyl or alkynyl; and the transition metal is selected from groups 8-11 of the Periodic Table.

In certain embodiments, the present invention relates to the aforementioned method and the attendant definitions, wherein Z represents independently for each occurrence alkenyl or alkynyl; and the transition metal is iron, cobalt, nickel, copper, ruthenium, rhodium, palladium, silver, iridium or platinum.

In certain embodiments, the present invention relates to the aforementioned method and the attendant definitions, wherein Z represents independently for each occurrence alkenyl or alkynyl; and the transition metal is silver.

In certain embodiments, the present invention relates to a method of catalyzing an acid-catalyzed chemical reaction to give a product, comprising the step of exposing a reactant mixture to a salt represented by formula I:
C⁺A⁻ I
wherein:
C⁺ represents an onium cation, and
A⁻ represents an anion of formula Ia: 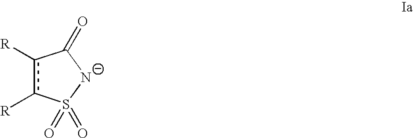
   wherein, independently for each occurrence:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈; or the two R taken together represent a substituted or unsubstituted fused cycloalkyl, cycloalkenyl, aryl, or heteroaryl ring;
   R₈ represents cycloalkyl, aryl, or heteroaryl;
   n represents an integer from 1-10 inclusive; and 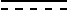
   represents a single or double bond; or
   an anion of formula Ib: 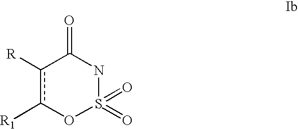
   wherein:
   R represents H, halide, alkyl, alkenyl, alkynyl, cycloaLky, aryl, heteroaryl, aralkyl, heteroaralkyl, hydroxy, amino, alkoxy, carboxyl, ester, keto, aldehyde, or —(CH₂)ₙ—R₈;
   R₁ represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R;
   or R and R₁ taken together represent a substituted or unsubstituted fused cycloalkyl, cycloalkenyl, aryl, or heteroaryl ring;
   R₈ represents cycloalkyl, aryl, or heteroaryl;
   n represents an integer from 1-10 inclusive; and 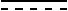
   represents a single or double bond; or
   an anion of formula Ic: 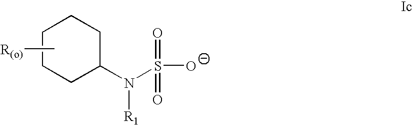
   wherein, independently for each occurrence:
   R is halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈, or any two adjacent R taken together represent a substituted or unsubstituted fused cycloalkyl, cycloalkenyl, aryl, or heteroaryl ring;
   R₁ is H, halide, alkyl, alkenyl, alkynyl, cycloalky, aryl, heteroaryl, aralkyl, heteroaralkyl, hydroxy, amino, alkoxy, carboxyl, keto, aldehyde, or —(CH₂)ₙ—R₈;
   R₈ represents cycloalkyl, aryl, or heteroaryl;
   n represents an integer from 1-10 inclusive; and
   o represents an integer from 0 to 11 inclusive; and wherein the onium cation has one of the following formulas: 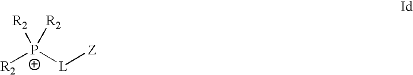
   wherein, independently for each occurrence:

R₂ represents alkyl, fluoroalkyl, cycloalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —CO₂R₂, —C(O)N(R″)₂, —C(O)N(R″)N(R″)₂, —N(R′)₂, —OR′, —SR′, —S(O)R″, —S(O)₂R″, —CN, —N(R″)P(O)(R)₂, —C(OR′)(R″)₂, alkenyl, or alkynyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
Ar represents aryl or heteroaryl;
J represents O, S, NR′, cycloalkyl, or heterocyclyl;
R₈ represents cycloalkyl, aryl, or heteroaryl;
m represents an integer from 1-10 inclusive; and
n represents an integer in the range 1-10 inclusive; or 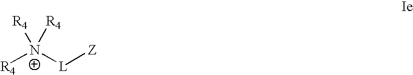
   wherein, independently for each occurrence:
R₄ represents alkyl, fluoroalkyl, cycloalkyl, aryl, heteroaryl, aralkyl, heteroaralkyl, or —(CH₂)ₙ—R₉; or ⁺N(R₄)₃ taken together represents pyridinium, imidazolium, benzimidazolium, pyrazolium, benzpyrazolium, indazolium, thiazolium, benzthiazolium, oxazolium, benzoxazolium, isoxazolium, isothiazolium, imdazolidenium, guanidinium, quinuclidinium, triazolium, tetrazolium, quinolinium, isoquinolinium, piperidinium, pyrrolidinium, morpholinium, pyridazinium, pyrazinium, piperazinium, triazinium, azepinium, or diazepinium;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —OC(O)R′, —CO₂R₄, —C(O)N(R″)₂, —C(O)N(R″)N(R″)₂, —N(R′)₂, —OR′, —SR′, —S(O)R″, —S(O)₂R″, —CN, —N(R″)P(O)(R₄)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₉;
R″ represents H, alkyl, fluoroalkyl, aryl, heteroaryl, aralkyl, heteroaralkyl, or —(CH₂)ₙ—R₉;
R₃ represents H, F, or alkyl;
R₈ represents cycloalkyl, aryl, or heteroaryl;
m represents an integer from 1-10 inclusive; and
n represents an integer from 1-10 inclusive; or 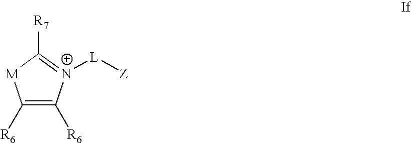
   wherein, independently for each occurrence:
M represents NR₅ or S;
R₅ represents H, alkyl, fluoroalkyl, cycloalkyl, aryl, heteroaryl, aralkyl, heteroaralkyl, or —(CH₂)ₙ—R₈; or R₅ is represented by formula If-a: 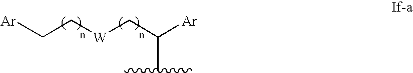
   wherein, independently for each occurrence:
   Ar represents a substituted or unsubstituted aryl or heteroaryl ring; and
   W represents O, NR₇, or S;
R₆ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R₇ represents H, alkyl, fluoroalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —CO₂R₅, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 0-10 inclusive; or 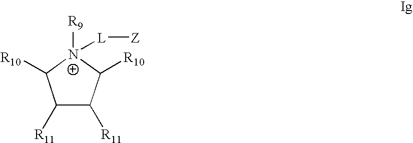
   wherein, independently for each occurrence:
R₉ represents alkyl, fluoroalkyl, cycloalkyl, aryl, heteroaryl, aralkyl, heteroaralkyl, or —(CH₂)ₙ—R₈;
R₁₀ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R₁₁ represents H, alkyl, fluoroalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —OC(O)R′—, —CO₂R₉, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 1-10 inclusive; or 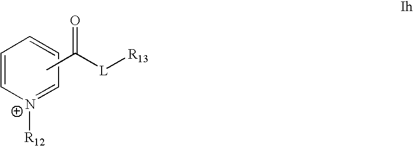
wherein:
L represents O, NR₁₂, or S;
R₁₂ represents H, alkyl, fluoroalkyl, aryl, heteroaryl, aralkyl, heteroaralkyl, or —(CH₂)ₙ—R₈;
R₁₃ represents H, alkyl, fluoroalkyl, aryl, heteroaryl, aralkyl, heteroaralkyl, or —(CH₂)ₙ—R₈; and
R₈ represents cycloalkyl, aryl, or heteroaryl.

In certain embodiments, the present invention relates to the aforementioned method and the attendant definitions, wherein Z represents independently for each occurrence —CO₂H.

In certain embodiments, the present invention relates to the aforementioned method and the attendant definitions, wherein said reactant mixture comprises an alcohol; and said product is an ether.

In certain embodiments, the present invention relates to the aforementioned method and the attendant definitions, wherein said reactant mixture comprises an alcohol and a carboxylic acid; and said product is an ester.

In certain embodiments, the present invention relates to the aforementioned method and the attendant definitions, wherein said reactant mixture comprises an ester and water; and said product is a carboxylic acid.

In certain embodiments, the present invention relates to the aforementioned method and the attendant definitions, wherein said reactant mixture comprises an alcohol and a first ester; and said product is a second ester.

In certain embodiments, the present invention relates to the aforementioned method and the attendant definitions, wherein said reactant mixture comprises a 1,2-diol; and said product is a ketone.

In certain embodiments, the present invention relates to the aforementioned method and the attendant definitions, wherein said reactant mixture comprises an alcohol; and said product is an alkene.

In certain embodiments, the present invention relates to the aforementioned method and the attendant definitions, wherein said reactant mixture comprises a first alkene; and said product is a second alkene.

In certain embodiments, the present invention relates to the aforementioned method and the attendant definitions, wherein said reactant mixture comprises a first aromatic compound and a nitrating agent; and said product is a second aromatic compound comprising a nitro group.

In certain embodiments, the present invention relates to the aforementioned method and the attendant definitions, wherein said reactant mixture comprises a first aromatic compound and an alcohol; and said product is a second aromatic compound comprising an alkyl group.

In certain embodiments, the present invention relates to the aforementioned method and the attendant definitions, wherein said reactant mixture comprises a first aromatic compound and a carboxylic acid; and said product is a second aromatic compound comprising an acyl group.

In certain embodiments, the present invention relates to the aforementioned method and the attendant definitions, wherein Z represents independently for each occurrence —CO₂H; and said reactant mixture comprises an alcohol; and said product is an ether.

In certain embodiments, the present invention relates to the aforementioned method and the attendant definitions, wherein Z represents independently for each occurrence —CO₂H; and said reactant mixture comprises an alcohol and a carboxylic acid; and said product is an ester.

In certain embodiments, the present invention relates to the aforementioned method and the attendant definitions, wherein Z represents independently for each occurrence —CO₂H; and said reactant mixture comprises an ester and water; and said product is a carboxylic acid.

In certain embodiments, the present invention relates to the aforementioned method and the attendant definitions, wherein Z represents independently for each occurrence —CO₂H; and said reactant mixture comprises an alcohol and a first ester; and said product is a second ester.

In certain embodiments, the present invention relates to the aforementioned method and the attendant definitions, wherein Z represents independently for each occurrence —CO₂H; and said reactant mixture comprises a 1,2-diol; and said product is a ketone.

In certain embodiments, the present invention relates to the aforementioned method and the attendant definitions, wherein Z represents independently for each occurrence —CO₂H; said reactant mixture comprises an alcohol; and said product is an alkene.

In certain embodiments, the present invention relates to the aforementioned method and the attendant definitions, wherein Z represents independently for each occurrence —CO₂H; said reactant mixture comprises a first alkene; and said product is a second alkene.

In certain embodiments, the present invention relates to the aforementioned method and the attendant definitions, wherein Z represents independently for each occurrence —CO₂H; said reactant mixture comprises a first aromatic compound and a nitrating agent; and said product is a second aromatic compound comprising a nitro group.

In certain embodiments, the present invention relates to the aforementioned method and the attendant definitions, wherein Z represents independently for each occurrence —CO₂H; said reactant mixture comprises a first aromatic compound and an alcohol; and said product is a second aromatic compound comprising an alkyl group.

In certain embodiments, the present invention relates to the aforementioned method and the attendant definitions, wherein Z represents independently for each occurrence —CO₂H; said reactant mixture comprises a first aromatic compound and a carboxylic acid; and said product is a second aromatic compound comprising an acyl group.

In certain embodiments, the present invention relates to a method of catalyzing a base-catalyzed chemical reaction to give a product, comprising the step of exposing a reactant mixture to a salt represented by formula I:
C⁺A⁻ I
wherein:
C⁺ represents an onium cation, and
A⁻ represents an anion of formula Ia: 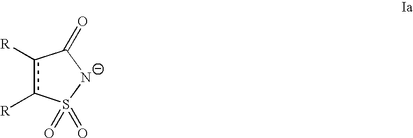
   wherein, independently for each occurrence:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈; or the two R taken together represent a substituted or unsubstituted fused cycloalkyl, cycloalkenyl, aryl, or heteroaryl ring;
   R₈ represents cycloalkyl, aryl, or heteroaryl;
   n represents an integer from 1-10 inclusive; and 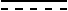
   represents a single or double bond; or
   an anion of formula Ib: 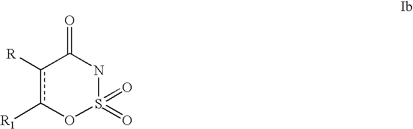
   wherein:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
   R₁ represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₉;
   or R and R₁ taken together represent a substituted or unsubstituted fused cycloalkyl, cycloalkenyl, aryl, or heteroaryl ring;
   R₈ represents cycloalkyl, aryl, or heteroaryl;
   n represents an integer from 1-10 inclusive; and 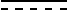
   represents a single or double bond; or
   an anion of formula Ic: 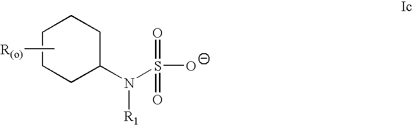
   wherein, independently for each occurrence:
   R is halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈, or any two adjacent R taken together represent a substituted or unsubstituted fused cycloalkyl, cycloalkenyl, aryl, or heteroaryl ring;
   R₁ is H, halide, alkyl, alkenyl, alkynyl, cycloalky, aryl, heteroaryl, aralkyl, heteroaralkyl, hydroxy, amino, alkoxy, carboxyl, keto, aldehyde, or —(CH₂)ₙ—R₈;
   R₈ represents cycloalkyl, aryl, or heteroaryl;
   n represents an integer from 1-10 inclusive; and
   o represents an integer from 0 to 11 inclusive; and wherein the onium cation has one of the following formulas: 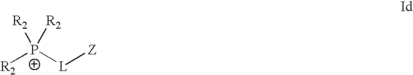
      wherein, independently for each occurrence:
   R₂ represents alkyl, fluoroalkyl, cycloalkyl, aryl, heteroaryl, aralkyl, heteroaralkyl, or —(CH₂)ₙ—R₈;
   L represents (C(R₃)₂)ₙ, (C(R₃)₂)ₙJ(C(R₃)₂)ₘ, or (C(R₃)₂)ₙAr(C(R₃)₂)ₘ;
   Z represents H, —CO₂H, —CO₂R₂, —C(O)N(R″)₂, —C(O)N(R″)N(R″)₂, —N(R′)₂, —OR′, —SR′, —S(O)R″, —S(O)₂R″, —CN, —N(R″)P(O)(R)₂, —C(OR′)(R″)₂, alkenyl, or alkynyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   Ar represents aryl or heteroaryl;
   J represents O, S, NR′, cycloalkyl, or heterocyclyl;
   R₈ represents cycloalkyl, aryl, or heteroaryl;
   m represents an integer from 1-10 inclusive; and
   n represents an integer in the range 1-10 inclusive; or 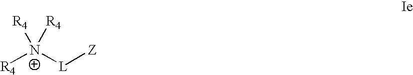
      wherein, independently for each occurrence:
   R₄ represents alkyl, fluoroalkyl, cycloalkyl, aryl, heteroaryl, aralkyl, heteroaralkyl, or —(CH₂)ₙ—R₈; or ⁺N(R₄)₃ taken together represents pyridinium, imidazolium, benzimidazolium, pyrazolium, benzpyrazolium, indazolium, thiazolium, benzthiazolium, oxazolium, benzoxazolium, isoxazolium, isothiazolium, imdazolidenium, guanidinium, quinuclidinium, triazolium, tetrazolium, quinolinium, isoquinolinium, piperidinium, pyrrolidinium, morpholinium, pyridazinium, pyrazinium, piperazinium, triazinium, azepinium, or diazepinium;
   L represents (C(R₃)₂)ₙ, (C(R₃)₂)ₙJ(C(R₃)₂)ₘ, or (C(R₃)₂)ₙAr(C(R₃)₂)ₘ;
   Z represents H, —CO₂H, —OC(O)R′, —CO₂R₄, —C(O)N(R″)₂, —C(O)N(R″)N(R″)₂, —N(R′)₂, —OR′, —SR′, —S(O)R″, —S(O)₂R″, —CN, —N(R″)P(O)(R₄)₂, —C(OR′)(R″)₂, alkenyl, or alkynyl;
   Ar represents aryl or heteroaryl;
   J represents O, S, NR′, cycloalkyl, or heterocyclyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   R₈ represents cycloalkyl, aryl, or heteroaryl;
   m represents an integer from 1-10 inclusive; and
   n represents an integer from 1-10 inclusive; or 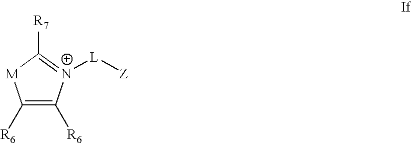
      wherein, independently for each occurrence:
   M represents NR₅ or S;
   R₅ represents H, alkyl, fluoroalkyl, cycloalkyl, aryl, heteroaryl, aralkyl, heteroaralkyl, or —(CH₂)ᵣ—R₈; or R₅ is represented by formula If-a: 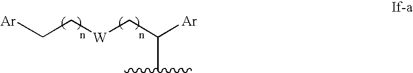
      wherein, independently for each occurrence:
      Ar represents a substituted or unsubstituted aryl or heteroaryl ring; and
      W represents O, NR₇, or S;
   R₆ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R₇ represents H, alkyl, fluoroalkyl, aryl, heteroaryl, aralkyl, heteroaralkyl, or —(CH₂)ₙ—R₈;
   L represents (C(R₃)₂)ₙ, (C(R₃)₂)ₙJ(C(R₃)₂)ₘ, or (C(R₃)₂)ₙAr(C(R₃)₂)ₘ;
   Z represents H, —CO₂H, —CO₂R₅, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
   Ar represents aryl or heteroaryl;
   J represents O, S, NR′, cycloalkyl, or heterocyclyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   R₈ represents cycloalkyl, aryl, or heteroaryl;
   m represents an integer from 1-10 inclusive; and
   n represents an integer from 0-10 inclusive; or 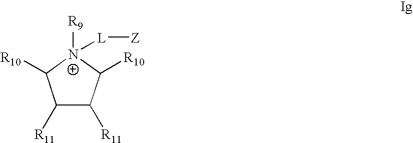
      wherein, independently for each occurrence:
   R₉ represents alkyl, fluoroalkyl, cycloalkyl, aryl, heteroaryl, aralkyl, heteroaralkyl, or —(CH₂)ₙ—R₈;
   R₁₀ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R₁₁ represents H, alkyl, fluoroalkyl, aryl, heteroaryl, aralkyl, heteroaralkyl, or —(CH₂)ₙ—R₈;
   L represents (C(R₃)₂)ₙ, (C(R₃)₂)ₙJ(C(R₃)₂)ₘ, or (C(R₃)₂)ₙAr(C(R₃)₂)ₘ;
   Z represents H, —CO₂H, —OC(O)R′—, —CO₂R₉, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
   Ar represents aryl or heteroaryl;
   J represents O, S, NR′, cycloalkyl, or heterocyclyl;
   R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
   R″ represents H, alkyl, fluoroalkyl, aryl, heteroaryl, aralkyl, heteroaralkyl, or —(CH₂)ₙ—R₈;
   R₃ represents H, F, or alkyl;
   R₈ represents cycloalkyl, aryl, or heteroaryl;
   m represents an integer from 1-10 inclusive; and
   n represents an integer from 1-10 inclusive; or 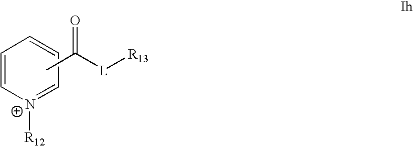
   wherein:
   L represents O, NR₁₂, or S; R₁₂ represents H, alkyl, fluoroalkyl, aryl, heteroaryl, aralkyl, heteroaralkyl, or —(CH₂)ₙ—R₈;
   R₁₃ represents H, alkyl, fluoroalkyl, aryl, heteroaryl, aralkyl, heteroaralkyl, or —(CH₂)ₙ—R₈; and
   R₈ represents cycloalkyl, aryl, or heteroaryl.

In certain embodiments, the present invention relates to the aforementioned method and the attendant definitions, wherein Z represents independently for each occurrence —N(R′)₂.

In certain embodiments, the present invention relates to a method of preparing a solution, comprising the step of combining a solute and a solvent to produce a solution, wherein said solvent is a salt represented by formula I:
C⁺A⁻ I
wherein:
C⁺ represents an onium cation, and
A⁻ represents an anion of formula Ia: 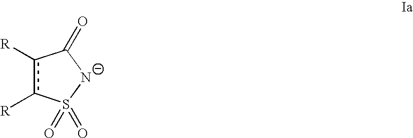
   wherein, independently for each occurrence:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈; or the two R taken together represent a substituted or unsubstituted fused cycloalkyl, cycloalkenyl, aryl, or heteroaryl ring;
   R₈ represents cycloalkyl, aryl, or heteroaryl;
   n represents an integer from 1-10 inclusive; and 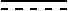
   represents a single or double bond; or
   an anion of formula Ib: 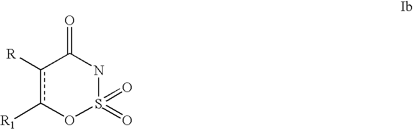
   wherein:
   R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
   R₁ represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
   or R and R₁ taken together represent a substituted or unsubstituted fused cycloalkyl, cycloalkenyl, aryl, or heteroaryl ring;
   R₈ represents cycloalkyl, aryl, or heteroaryl;
   n represents an integer from 1-10 inclusive; and 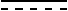
   represents a single or double bond; or
   an anion of formula Ic: 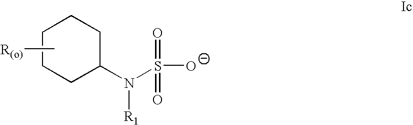
   wherein, independently for each occurrence:
   R is halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈, or any two adjacent R taken together represent a substituted or unsubstituted fused cycloalkyl, cycloalkenyl, aryl, or heteroaryl ring;
   R₁ is H, halide, alkyl, alkenyl, alkynyl, cycloalky, aryl, heteroaryl, aralkyl, heteroaralkyl, hydroxy, amino, alkoxy, carboxyl, keto, aldehyde, or —(CH₂)ₙ—R₈;
   R₈ represents cycloalkyl, aryl, or heteroaryl;
   n represents an integer from 1-10 inclusive; and
   o represents an integer from 0 to 11 inclusive; and wherein the onium cation has one of the following formulas: 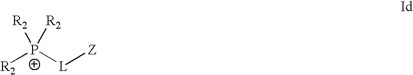
      wherein, independently for each occurrence:

R₂ represents alkyl, fluoroalkyl, cycloalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —CO₂R₂, —C(O)N(R″)₂, —C(O)N(R″)N(R″)₂, —N(R′)₂, —OR′, —SR′, —S(O)R″, —S(O)₂R″, —CN, —N(R″)P(O)(R)₂, —C(OR′)(R″)₂, alkenyl, or alkynyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
Ar represents aryl or heteroaryl;
J represents O, S, NR′, cycloalkyl, or heterocyclyl;
R₈ represents cycloalkyl, aryl, or heteroaryl;
m represents an integer from 1-10 inclusive; and
n represents an integer in the range 1-10 inclusive; or 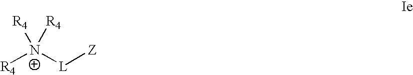
   wherein, independently for each occurrence:

R₄ represents alkyl, fluoroalkyl, cycloalkyl, aryl, heteroaryl, aralkyl, heteroaralkyl, or —(CH₂)ₙ—R₈; or ⁺N(R₄)₃ taken together represents pyridinium, imidazolium, benzimidazolium, pyrazolium, benzpyrazolium, indazolium, thiazolium, benzthiazolium, oxazolium, benzoxazolium, isoxazolium, isothiazolium, imdazolidenium, guanidinium, quinuclidinium, triazolium, tetrazolium, quinolinium, isoquinolinium, piperidinium, pyrrolidinium, morpholinium, pyridazinium, pyrazinium, piperazinium, triazinium, azepinium, or diazepinium;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —OC(O)R′, —CO₂R₄, —C(O)N(R″)₂, —C(O)N(R″)N(R″)₂, —N(R′)₂, —OR′, —SR′, —S(O)R″, —S(O)₂R″, —CN, —N(R″)P(O)(R₄)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 1-10 inclusive; or 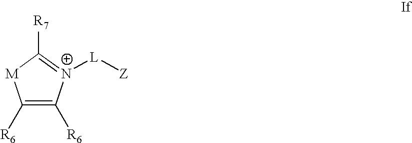
   wherein, independently for each occurrence:
M represents NR₅ or S;
R₅ represents H, alkyl, fluoroalkyl, cycloalkyl, aryl, heteroaryl, aralkyl, heteroaralkyl, or —(CH₂)ₙ—R₈; or R₅ is represented by formula If-a: 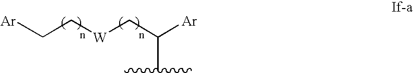
   wherein, independently for each occurrence:
   Ar represents a substituted or unsubstituted aryl or heteroaryl ring; and
   W represents O, NR₇, or S;
R₆ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R₇ represents H, alkyl, fluoroalkyl, aryl, heteroaryl, aralkyl, heteroaralkyl, or —(CH₂)ₙ—R₈;
L represents (C(R₃)₂)ₘ, (C(R₃)₂)ₙJ(C(R₃)₂)ₘ, or (C(R₃)₂)ₙAr(C(R₃)₂)ₘ;
Z represents H, —CO₂H, —CO₂R₅, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₉;
R₃ represents H, F, or alkyl;
R₈ represents cycloalkyl, aryl, or heteroaryl;
m represents an integer from 1-10 inclusive; and
n represents an integer from 0-10 inclusive; or 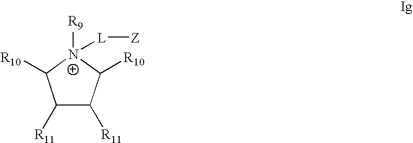
   wherein, independently for each occurrence:
R₉ represents alkyl, fluoroalkyl, cycloalkyl, aryl, heteroaryl, aralkyl, heteroaralkyl, or —(CH₂)ₙ—R₈;
R₁₀ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R₁₁ represents H, alkyl, fluoroalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —OC(O)R′—, —CO₂R₉, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 1-10 inclusive; or 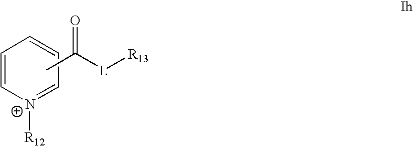
wherein:
L represents O, NR₁₂, or S;
R₁₂ represents H, alkyl, fluoroalkyl, aryl, heteroaryl, aralkyl, heteroaralkyl, or —(CH₂)ₙ—R₈;
R₁₃ represents H, alkyl, fluoroalkyl, aryl, heteroaryl, aralkyl, heteroaralkyl, or —(CH₂)ₙ—R₈; and
R₈ represents cycloalkyl, aryl, or heteroaryl.
   Applications of Ionic Liquids

Because of their unique properties and the ability to fine tune an ionic liquid to a particular need, ionic liquids have a wide array of applications. An incomplete list of beneficial properties of ionic liquids includes: no vapor pressure, reasonable thermal stability, good solubility for organic and organometallic compounds, gas solubility (CO, O₂, H₂, and the like) is good, can be immiscible with alkanes, tunable solvent properties (solubility, polarity, etc.), non-coordinating solvent, electrically conducting, low viscosity, low toxicology, good electrochemical stability, and lipophilic room temperature ionic liquids can be used with aqueous biphasic systems.

Ionic liquids have been used in a number of broad and varied areas including the following non-limiting examples:
Energy, which encompasses batteries, fuel cells, photovoltaic cells, heat storage (based on the large evolution of heat upon crystallization), and supercapS;
Coatings, which encompasses metal depositions, analytic, lubricants, and surfactantS;
Chemical, which encompasses organic synthesis, chiral synthesis, polymerization, and catalysiS;
Biotechnology, which encompasses enzyme reactions and purification of proteinS;
Chemical Engineering, which encompasses extractions, separations, membranes, and extractive distillationS;
Other, which encompasses light emitting electrochemical cells (LECs), liquid crystals, nano particles, artificial muscles, oils/advanced fluids, and electrosynthesis of conducting polymers.

Ionic liquids that preferentially dissolve certain gaseous species can be used in conventional gas absorption applications. The non-volatile nature of ionic liquids plays two important roles. First, there will be no cross-contamination of the gas stream by the solvent during operation. This means no solvent loss and no air pollution. Second, regeneration of the solvent is easy; a simple flash or mild distillation step is all that is required to remove the gas from the solvent, again with no cross-contamination.

In addition to their use as conventional absorbents, ionic liquids may be immobilized on a support and used in a supported liquid membrane (SLM). The membrane will work if a gas preferentially dissolves in the liquid. SLMs may be used in a continuous separation process without a regeneration step. Conventional SLM technology is undermined by the fact that the liquid in which the gas dissolves eventially evaporates, thus rendering the membrane useless. Since ionic liquids are completely non-volatile, this problem is eliminated.

Ionic liquids also find use in the conversion of brown coal and oil shale into value-added products, such as alternative synthetic fuels and/or high-quality chemical feedstocks. For example, 1-butyl-3-methyl imidazolium, has been used to extract organic compounds from Estonian oil shale kerogen at various temperatures. Results at 175° C. yielded soluble products with an increase of ten times over that obtained using conventional organic solvents.

Bronsted-acidic ILs also act as proton shuttles, functionally carrying protons from acidic resin surfaces (e.g., Nafion) to the surrounding medium, where they are more free to react than if the proton is held at the polymer surface. Moreover, the Bronsted-acidic ILs have absolutely no vapor pressure when dissolved in water. For example, a relatively concentrated solution of HCl gives off HCl gaS; in contrast, a Bronsted-acidic IL gives off no gaseous acid—pH paper suspended above the surface does not change colors!

Many product streams, particularly in the field of petroleum chemistry, include olefins and non-olefins. For example, ethane crackers tend to produce a mixture of ethane and ethylene. The ethylene is typically separated from the ethane via distillation. Because the boiling points of ethylene and ethane are relatively close to one another, the distillation is typically done at very low temperatures and/or high pressureS; the separation is relatively expensive. The same problems are observed when separating propane from propylene in dehydrogenation facilities. Ionic liquids are useful is separating such mixtures. For example, an ionic liquid with a pendant functional group that coordinates the pi-biond of an olefin may be used to dissolve selectively the olefinic components of such a mixture. Likewise, an ionic liquid with a pendant functional group that coordinates a transition metal capable of coordinating the pi-bond of an olefin may be used to dissolve selectively the olefinic components of such a mixture. In either case, the dissolved olefins subsequently can be isolated by desorption.

The chemical field has made good use of ionic liquids where the potential for even greater use is constantly being explored. Known chemical reactions carried out in ionic liquids include butene oligomerization, hydrodimerization of dienes, alkylation of olefins, hydrogenation (e.g. of cyclohexene), hydroformylation, oxidation (e.g. epoxidation of 2,2-dimethyl chromene), alkoxycarbonylation (of styrene), and hydrodimerizations/telomerizations (e.g. of 1,3-butadiene). An advantage to using ionic liquids as the medium for chemical reactions is that the rates and selectivities can be modified by modifying the ionic liquid. Reaction mechanisms are similar to those in organic solvents.

In particular, research published since the early 1990's points to significant opportunities to replace solution polymerizations using VOCs with ionic liquids. Polymerizations that have been carried out in ionic liquid mediums include homopolymerizations with faster rates and higher MW; living radical homopolymerization where the catalyst has been retained in the ionic liquid phase; statistical copolymerization which may create copolymers having monomer sequences not readily achievable using conventional solventS; and block copolymerization where ionic liquid routes may simplify, reduce cost of producing block copolymers with defined structures. In another related area, polymer-ionic liquid composites as new possible materials have been explored.

Separations is another area in particular that is making use of ionic liquids. Highlights in this area include liquid extractions of organics and metals from aqueous solutionS; sulfur removal and slective separations by solubility, extractive distillation, etc. in hydrocarbon processing; gas separations where task-specific ionic liquids have been developed based on solubilitieS; solvent regeneration as applied to, for example, supercritical fluids, pervaporation, and distillationS; supported liquid membraneS; electrorefining; and analytical separations.

Significant academic and industrial interest has also been directed towards using ionic liquids in fuel technology with the potential for high-volume, energy saving applications. Highlights in this area include liquefaction, gasifaction, and chemical modification of solid fuels (e.g. coal, oil shale, kerogen, and the like); sweetening of sour gaS; optimization for high-octane fuel additiveS; environmental removal of contaminants from waste streamS; desulfurization of fossil fuelS; and safer and more efficient nuclear fuel cycles.

### Exemplification

The invention now being generally described, it will be more readily understood by reference to the following examples, which are included merely for purposes of illustration of certain aspects and embodiments of the present invention, and are not intended to limit the invention.

General Considerations

¹H NMR (300 MHz) and ¹³C NMR (75 MHz) spectra were obtained as solutions in either CDCl₃ or D₂O. Chemical shifts were reported in parts per million (ppm, δ) and referenced to CHCl₃ (δ 7.27) or D₂O (δ 4.88). Infrared spectra were recorded as a thin film on sodium chloride and absorptions were reported in wavenumbers (cm⁻¹). Melting points are uncorrected. Distillations were performed using a Kugelrohr ball-tube distillation apparatus. Gas chromatographic analyses were performed using an Agilent 6850 system (FID). TLC analyses were performed on Whatman flexible polyester backed TLC plates with a fluorescent indicator. Detection was conducted by UV absorption (254 nm) and charring with 10% KMnO₄ in water. Baker silica gel (47-61 microns) was used for all chromatographic separations. Anhydrous organic solvents were dried and then distilled prior to use. Acetic acid, acetic anhydride, benzopinacole, ethanol, hexanoic acid, 1-octanol, pinacol and p-toluenesulfonic acid were not purified prior to use. All other chemicals used for synthetic procedures were reagent grade or better. Solutions were concentrated in vacuo with a rotary evaporator and the residue was purified using a silica gel column unless specified otherwise.

### Example 1

Synthesis of [HMIM] Ace

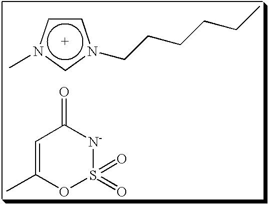

In a 500 mL round-bottomed flask charged with a magnetic stir bar, 25.0 g of potassium acesulfame (0.12 mol) is added to 200 mL of acetone. To this solution/suspension is added in one portion 21.0 g (0.10 mol) of 1-hexyl-3-methyl imidazolium chloride, [HMIM]Cl. The mixture was stirred overnight, after which time precipitated KCl was removed by filtration. The acetone solution was then evaporated, extracted with chloroform (200 mL) and re-filtered to remove residual KCl and unreacted potassium acesulfame. Removal of the solvent in vacuo provided the final product as a yellow oil (30.6 g, 93%). Using similar quantities of reagents, water or alcohols such as ethanol or methanol may be used with similar results. In the case of water as a solvent, the first filtration step is omitted (no initial precipitation of KCl) and the water is directly evaporated, producing a residue which is extracted into chloroform, filtered and evaporated again to produce the product. This procedure is generally applicable to the synthesis of the Acesulfame derived ionic liquids, and may be accomplished using any of the halide salts of the initial onium cation (chloride, bromide or iodide). Those skilled in the art will also appreciate that other salts of the desired acesulfame anion (e.g., sodium) will suffice in the synthesis as well.

In the event that a completely chloride-free salt is required, the following modifications are made: Equimolar amounts of the silver salt of acesulfame and a halide salt (chloride, bromide or iodide) are combined in water in a vessel protected from light, instantaneously producing a precipitate of the corresponding silver halide salt. After stirring for 15 min, the suspension is filtered to remove the precipitated silver salt. The water is removed in vacuo, producing a residue which is then extracted with chloroform, re-filtered and re-evaporated, providing the desired acesulfame salt in quantitative yield. In the event that traces of silver ion persist (as indicated by a darkening in color of the ionic liquid over time upon exposure to light), the system is allowed to remain in bright light for a period of one to two weeks, after which point it is dissolved in chloroform, passed through a short plug of basic alumina or silica gel and re-evaporated.

¹H-NMR data for [HMIM]Ace (hexylmethylimidazolium acesulfamate, above). 300 MHz, CDCl₃, δ. 0.73 (t, 3H, CH₃); 1.17 (overlapping d, 6H, CH₂); 1.73 (m, 2H, CH₂); 1.89 (s, 3H, CH₃); 3.89 (s, 3H, CH₃); 4.11 (t, 2H, CH₂); 5.33 (s, 1H, CH); 7.31 (m, 1H, CH); 7.37 (m, 1H, CH); 9.47 (m, 1H, CH).

### Example 2

Synthesis of [HMIM] Sac

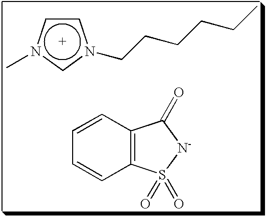

In a 500 mL round-bottomed flask charged with a magnetic stir bar, 18.2 g of sodium saccharin (0.088 mol) is added to 200 mL of acetone. To this solution/suspension is added in one portion 17.2 g (0.085 mol) of 1-hexyl-3-methyl imidazolium chloride, [HMIM]Cl. The mixture was stirred overnight, after which time precipitated NaCl was removed by filtration. The acetone solution was then evaporated, extracted with choloroform (200 mL) and re-filtered to remove residual NaCl and unreacted sodium saccharin. Removal of the solvent in vacuo provided the final product as a golden oil (28.2 g, 95%). Using similar quantities of reagents, water or alcohols such as ethanol or methanol may be used with similar results. In the case of water as a solvent, the first filtration step is omitted (no initial precipitation of NaCl) and the water is directly evaporated, producing a residue which is extracted into chloroform, filtered and evaporated again to produce the product. This procedure is generally applicable to the synthesis of the Saccharin derived ionic liquids, and may be accomplished using any of the halide salts of the initial onium cation (chloride, bromide or iodide). Those skilled in the art will also appreciate that other salts of the desired saccharine anion (e.g., potassium) will suffice in the synthesis as well.

In the event that a completely chloride-free salt is required, the following modifications are made: Equimolar amounts of the silver salt of saccharin and a halide salt (chloride, bromide or iodide) are combined in water in a vessel protected from light, instantaneously producing a precipitate of the corresponding silver halide salt. After stirring for 15 min, the suspension is filtered to remove the precipitated silver salt. The water is removed in vacuo, producing a residue which is then extracted with chloroform, re-filtered and re-evaporated, providing the desired saccharin salt in quantitative yield. In the event that traces of silver ion persist (as indicated by a darkening in color of the ionic liquid over time upon exposure to light), the system is allowed to remain in bright light for a period of one to two weeks, after which point it is dissolved in chloroform, passed through a short plug of basic alumina or silica gel and re-evaporated.

¹H-NMR data for [HMIM]Sac (hexylmethylimidazolium saccharinate, above). 300 MHz, CDCl₃, δ. 0.68 (t, 3H, CH₃); 1.09 (overlapping d, 6H, CH₂); 1.68 (m, 2H, CH₂); 3.90 (s, 3H, CH₃); 4.10 (t, 2H, CH₂); 7.29 (m, 1H, CH); 7.39 (m, 1H, CH); 7.44 (overlapping m, 2H, CH); 7.60 (m, 1H, CH); 7.64 (m, 1H, CH); 9.58 (m, 1H, CH).

### Example 3

Synthesis of [HMIM] Cyc

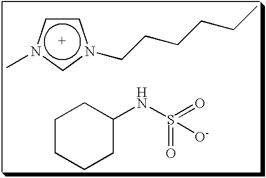

In a 500 mL round-bottomed flask protected from visible light and charged with a magnetic stir bar, 10.0 g of silver cyclamate (0.035 mol) is added to 250 mL of water. To this solution/suspension is added in one portion 7.1 g (0.035 mol) of 1-hexyl-3-methyl imidazolium chloride, [HMIM]Cl. The mixture is stirred for 3 hours, during which time the consistency and color of the suspended solid changed. After the stirring period, the precipitated AgCl was removed by filtration. The aqueous solution was then evaporated, the residue extracted with methanol (200 mL) and re-filtered to remove residual AgCl or unreacted silver cyclamate. Removal of the methanol in vacuo provided the final product as a pale yellow oil (10.6 g, 88%). In the event that traces of silver ion persist (as indicated by a darkening in color of the ionic liquid over time upon exposure to light), the system is allowed to remain in bright light for a period of one to two weeks, after which point it is dissolved in methanol, passed through a short plug of basic alumina or silica gel and re-evaporated. The use of the silver salt of cyclamate is preferred for the synthesis of cyclamate based ionic liquids, although other salts such as the sodium and potassium salts may be used (while generally providing poorer ion exchange and lower yields of pure product).

¹H-NMR data for [HMIM]Cyc (hexylmethylimidazolium cyclamate, above). 300 MHz, CDCl₃, δ. 0.80 (t, 3H, CH₃); 1.00-1.30 (overlapping multiplets, 10H, CH₂); 1.49 (m, 2H, CH₂); 1.62 (m, 2H, CH₂); 1.82 (m, 2H, CH₂); 2.04 (m, 2H, CH₂); 3.16 (m, 1H); 3.99 (s, 3H, CH₃); 4.20 (t, 2H, CH₂); 7.36 (s, 1H, CH); 7.53 (s, 1H, CH); 9,81 (s, 1H, CH).

### Example 4

Synthesis of [HDMIM] Sac

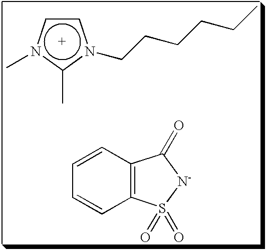

[HDMIM]Saccharinate was prepared in the same manner as in Example 2 except 1-hexyl-2,3-dimethyl imidazolium chloride [HDMIM]Cl was used instead of [HMIM]C.

¹H-NMR data for [HDMIM]Sac (hexyldimethylimidazolium saccharinate, above). 300 MHz, CDCl₃, δ. 0.66 (t, 3H, CH₃); 1.07 (overlapping d, 6H, CH₂); 1.55 (m, 2H, CH₂); 2.51 (s, 3H, CH3); 3.74 (s, 3H, CH₃); 3.92 (t, 2H, CH₂); 7.21 (m, 1H, CH); 7.35 (overlapping m, 2H, CH); 7.39 (m, 1H, CH); 7.46 (m, 1H, CH); 7.51 (m, 1H, CH).

### Example 5

Synthesis of [butyryl choline]Cyc

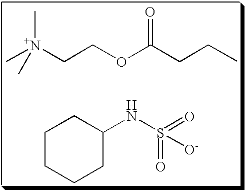

[butyryl choline] Cyclamate was prepared in the same manner as in Example 3 except butyryl choline chloride was used instead of [HMIM]Cl.

¹H-NMR data for [butyryl choline]Cyclamate (above). 300 MHz, CDCl₃, δ. 0.91 (t, 3H, CH3); 1.04-1.32 (overlapping m, 6H, CH₂); 1.50-1.70 (overlapping m, 5H, CH, CH₂); 2.03 (m, 2H, CH₂); 2.30 (t, 2H, CH₂); 3.11 (m, 1H); 3.37 (s, 9H, CH₃); 3.93 (m, 2H, CH₂); 4.51 (m, 2H, CH₂).

### Example 6

Synthesis of [butyryl choline]Sac

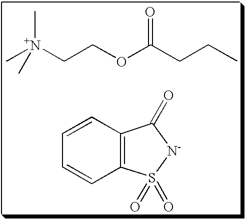

[butyryl choline]Saccharinate was prepared in the same manner as in Example 2 except butyryl choline chloride was used instead of [HMIM]Cl.

¹H-NMR data for [butyryl choline]Saccharinate (above). 300 MHz, CDCl₃, δ. 0.88 (t, 3H, CH₃); 1.55 (m, 2H, CH₂); 2.22 (m, 2H, CH₂); 3.40 (s, 9H, CH₃); 3.94 (m, 2H, CH₂) 4.51 (m, 2H, CH₂); 7.55 (m, 2H, CH); 7.73 (m, 2H, CH).

### Example 7

Synthesis of [butyryl choline]Ace

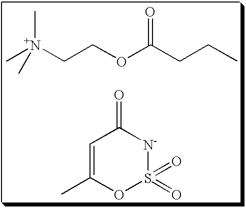

[butyryl choline]Acesulfamate was prepared in the same manner as in Example 1 except butyryl choline chloride was used instead of [HMIM]Cl.

¹H-NMR data for [butyryl choline]Acesulfamate (above). 300 MHz, CDCl₃, δ. 0.93 (t, 3H, CH₃); 1.62 (m, 2H, CH₂); 2.03 (s, 3H, CH₃); 2.31 (m, 2H, CH₂); 3.37 (s, 9H, CH₃); 3.87 (m, 2H, CH₂) 4.51 (m, 2H, CH₂); 5.50 (s, 1H, CH).

### Example 8

Synthesis of [tetra-n-butylphosphonium]Sac

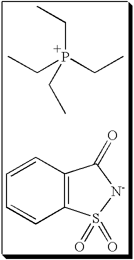

[tetra-n-butyl phosphonium]Saccharinate was prepared in the same manner as in Example 2 except tetra-n-butyl phosphonium chloride was used instead of [HMIM]Cl.

¹H-NMR data for [tetra-n-butyl phosphonium]Saccharinate (above). 300 MHz, CDCl₃, δ. 0.68 (t, 12H, CH₃); 1.24 (overlapping m, 16H, CH₂); 2.01 (overlapping m, 8H, CH₂); 7.47 (m, 2H, CH); 7.56 (m, 2H, CH).

### Example 9

Synthesis of [tetraethylammonium]Ace

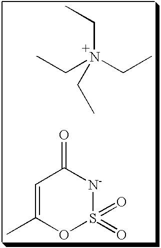

[tetraethylammonium]Acesulfamate was prepared in the same manner as in Example 1 except tetraethylammonium chloride was used instead of [HMIM]chloride.

¹H-NMR data for [tetraethylammonium]Acesulfamate (above). 300 MHz, CDCl₃, δ. 1.28 (t, 12H, CH₃); 1.95 (s, 3H, CH₃); 3.03 (q, 8H, CH₂); 5.37 (s, 1H, CH).

### Example 10

Synthesis of [tetraethylammonium]Sac

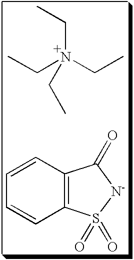

[tetraethylammonium]Saccharinate was prepared in the same manner as in Example 2 except tetraethylammonium chloride was used instead of [HMIM]Cl.

¹H-NMR data for [tetraethylammonium]saccharinate (above). 300 MHz, CDCl₃, δ. 1.28 (t, 12H, CH₃); 3.32 (q, 8H, CH₂); 7.52 (m, 2H, CH); 7.69 (m, 1H, CH); 7.76 (m, 1H, CH).

### Example 11

Synthesis of [methyltriethylammonium]Ace

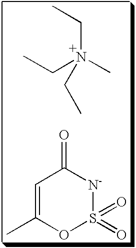

[methyltrietylammonium]Acesulfamate was prepared in the same manner as in Example 1 except methyltriethylammonium chloride was used instead of [HMIM]Cl.

¹H-NMR data for [methyltriethylammonium]Acesulfamate (above). 300 MHz, CDCl₃, δ. 1.23 (t, 9H, CH₃); 1.88 (s, 3H, CH₃); 2.97 (s, 3H, CH₃); 3.34 (q, 6H, CH₂); 5.31 (s, 1H, CH).

### Example 12

Synthesis of [methyltriethylammonium]Sac

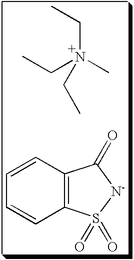

[methyltriethylammonium]Saccharinate was prepared in the same manner as in Example 2 except methyltriethylammonium chloride was used instead of [HMIM]Cl.

¹H-NMR data for [methyltriethylammonium]Saccharinate (above). 300 MHz, CDCl₃, δ. 1.24 (t, 9H, CH₃); 3.01 (s, 3H, CH₃); 3.34 (q, 6H, CH₂); 7.50 (overlapping m, 2H, CH); 7.65 (m, 1H, CH); 7.71 (m, 1H, CH).

### Example 13

Econazole is a representative of the “azole” family of antifungal drugs. They are usually used as their nitrate salts. The acesulfame and saccharinate salts are both IL (mp<100° C.) and they both have considerably improved solubilities in organic media. As a consequence, they might have improved properties for incorporation into topical creams. In addition, the apparent rates of dissolution of these salts in water is different from that of the nitrate salts, potentially altering their pharmacokinetic properties.
Synthesis of [econazolium]Ace

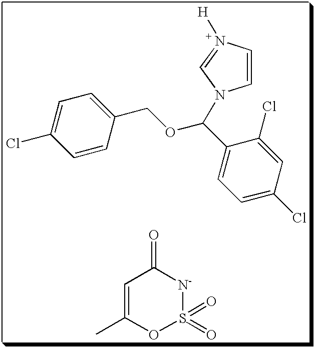

[econazolium]Acesulfamate was prepared in the same manner as in Example 1 except econazolium chloride was used instead of [HMIM]Cl.

¹H-NMR data for [econazolium]Ace (above). 300 MHz, CDCl₃, δ. 2.06 (s, 3H, CH₃); 4.10-4.61 (overlapping m, 3H); 4.97 (s, 1H); 5.58 (s, 1H); 6.92-7.34 (overlapping m, 8H, CH); 7.45 (m, 1H, CH); 8.57 (m, 1H, CH).

### Example 14

Synthesis of [econazolium]Sac

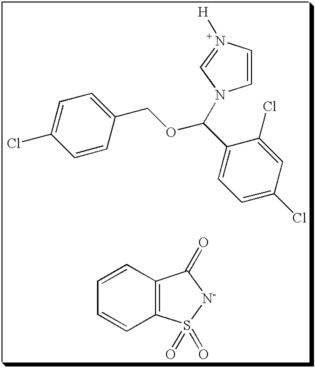

[econazolium]Saccharinate was prepared in the same manner as in Example 2 except econazolium chloride was used instead of [HMIM]Cl.

¹H-NMR data for [econazolium] Sac (above). 300 MHz, CDCl₃, δ. 4.17-4.53 (overlapping m, 3H); 5.00 (m, 1H); 6.97-7.34 (overlapping m, 8H, CH); 7.47 (m, 1H, CH); 7.67 (m, 2H, CH); 7.86 (m, 2H, CH); 8.62 (m, 1H, CH).

### Example 15

Synthesis of [trihexyl tetradecylphosphonium]Sac

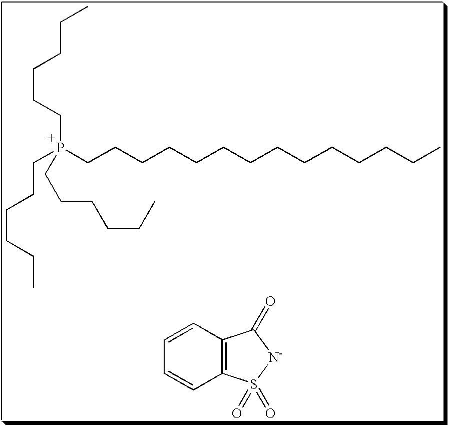

[trihexyl tetradecylphosphonium]Saccharinate was prepared in the same manner as in Example 2 except trihexyl tetradecylphosphonium chloride was used instead of [HMIM]Cl.

¹H-NMR data for [trihexyl tetradecylphosphonium]Saccharinate (above). 300 MHz, CDCl₃, δ. 0.82 (overlapping t, 12H, CH₃); 1.24 (overlapping m, 36H, CH₂); 1.46 (overlapping m, 12H, CH₂); 2.30 (overlapping m, 8H, CH₂); 7.49 (m, 2H, CH); 7.71 (m, 1H, CH); (7.80 (m, 1H, CH).

### Example 16

Butyl nicotinate is an ester of the vitamin nicotinic acid. The metabolic elimination product of the metabolism of nicotinic acid is the N-methylated nicotinate zwitterion. Given the forgoing, the cation below is likely to be physiologically and environmentally innocuous, as are salts of it with the sweeteners.
Synthesis of [butyl nicotinate]Sac

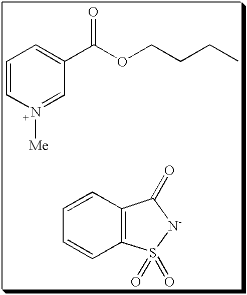

[butyl nicotinate]Saccharinate was prepared in the same manner as in Example 2 except butyl nicotinate chloride was used instead of [HMIM]Cl.

### Example 17

Synthesis of [butyl nicotinate]Ace

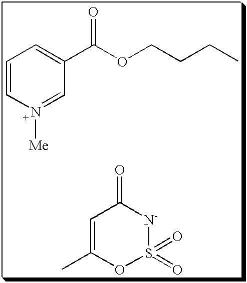

[butyl nicotinate]Acesulfamate was prepared in the same manner as in Example 1 except butyl nicotinate chloride was used instead of [HMIM]Cl.

¹H-NMR data for [N-methyl butyl nicotinate]Ace (above). 300 MHz, CDCl₃, δ. 0.97 (t, 3H, CH₃); 1.44 (m, 2H, CH₂); 1.73 (m, 2H, CH₂); 1.98 (s, 3H, CH₃); 4.39 (t, 2H, CH₂); 4.59 (s, 3H, CH₃); 5.44 (s, 1H); 8.19 (dd, 1H, CH); 8.84 (d, 1H, CH); 9.26 (s, 1H, CH); 9.29 (d, 1H, CH).

### Example 18

Synthesis of [1-hexyl-4-methyl thiazolium]Ace

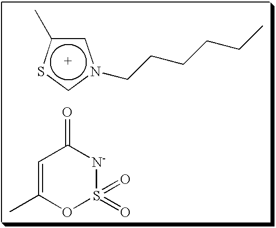

[1-hexyl-4-methyl thiazolium]Acesulfamate was prepared in the same manner as in Example 1 except 1-hexyl-4-methyl thiazolium chloride was used instead of [HMIM]Cl.

¹H-NMR data for [1-hexyl-4-methyl thiazolium]Ace (above). 300 MHz, CDCl₃, δ. 0.87 (t, 3H, CH₃); 1.31 (overlapping d, 6H, CH₂); 1.94 (m, 2H, CH₂); 2.12 (s, 3H, CH₃); 2.59 (s, 3H, CH₃); 4.46 (t, 2H, CH₂); 5.66 (s, 1H, CH); 8.04 (s, 1H, CH); 10.19 (s, 1H, CH).

### Incorporation by Reference

All of the U.S. patents and U.S. patent application publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

1. A salt represented by formula I:
C⁺A⁻ I
wherein:
C⁺ represents an onium cation, and
A⁻ represents an anion of formula Ia: 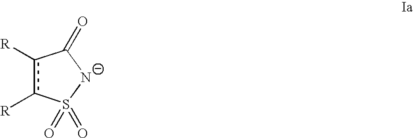
wherein, independently for each occurrence:
R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈; or the two R taken together form a substituted or unsubstituted fused cycloalkyl, cycloalkenyl, aryl, or heteroaryl ring;
R₈ represents cycloalkyl, aryl, or heteroaryl;
n represents an integer from 1-10 inclusive; and
\overline{---} represents a single or double bond; or
an anion of formula Ib: 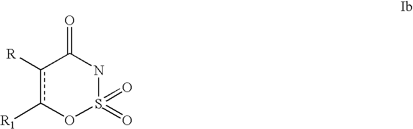
wherein:
R represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
R₁ represents H, halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈;
or R and R₁ taken together form a substituted or unsubstituted fused cycloalkyl, cycloalkenyl, aryl, or heteroaryl ring;
R₈ represents cycloalkyl, aryl, or heteroaryl;
n represents an integer from 1-10 inclusive; and
\overline{---} represents a single or double bond; or
an anion of formula Ic: 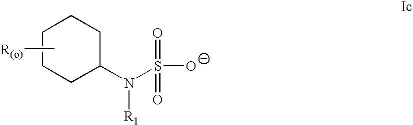
wherein, independently for each occurrence:
R is halide, alkyl, alkenyl, alkynyl, cycloalky, aryl, heteroaryl, aralkyl, heteroaralkyl, hydroxy, amino, alkoxy, carboxyl, ester, keto, aldehyde, or —(CH₂)ₙ—R₈, or any two adjacent R taken together form a substituted or unsubstituted fused cycloalkyl, cycloalkenyl, aryl, or heteroaryl ring;
R₁ is H, halide, alkyl, alkenyl, alkynyl, cycloalky, aryl, heteroaryl, aralkyl, heteroaralkyl, hydroxy, amino, alkoxy, carboxyl, keto, aldehyde, or —(CH₂)ₙ—R₉;
R₈ represents cycloalkyl, aryl, or heteroaryl;
n represents an integer from 1-10 inclusive; and
o represents an integer from 0 to 11 inclusive.

2. The salt of claim 1, wherein A⁻ is an anion of formula Ia.

3. The salt of claim 1, wherein A⁻ is an anion of formula Ia, wherein the two R groups taken together form a fused benzene ring.

4. The salt of claim 1, wherein A⁻ is an anion of formula Ib.

5. The salt of claim 1, wherein A⁻ is an anion of formula Ib, wherein \overline{---} represents a double bond, R represents H, and R₁ represents methyl.

6. The salt of claim 1, wherein A⁻ is an anion of formula Ic.

7. The salt of claim 1, wherein A⁻ is an anion of formula Ic, wherein o is 0 and R₁ is H.

8. The salt of claim 1, wherein the onium cation is selected from the following: phosphonium, ammonium, imidazonium, pyrrolidinium, pyridinium, thiazolium, arsonium, stibonium, oxonium, sulfonium, selenonium, telluronium, fluoronium, chloronium, bromonium, or iodonium cation.

9. The salt of claim 1, wherein the onium cation is an ammonium, imidazonium, phosphonium, pyridinium, pyrrolidinium, or thiazolium cation.

10. The salt of claim 1, wherein the onium cation is represented by formula Id: 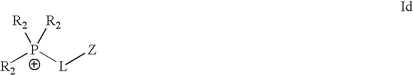
wherein, independently for each occurrence:
R₂ represents alkyl, fluoroalkyl, cycloalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —CO₂R₂, —C(O)N(R″)₂, —C(O)N(R″)N(R″)₂, —N(R′)₂, —OR′, —SR′, —S(O)R″, —S(O)₂R″, —CN, —N(R″)P(O)(R)₂, —C(OR′)(R″)₂, alkenyl, or alkynyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
Ar represents aryl or heteroaryl;
J represents O, S, NR′, cycloalkyl, or heterocyclyl;
R₈ represents cycloalkyl, aryl, or heteroaryl;
m represents an integer from 1-10 inclusive; and
n represents an integer in the range 1-10 inclusive.

11. The salt of claim 1, wherein the onium cation is represented by formula Ie: 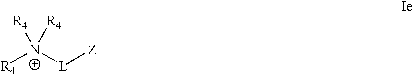
wherein, independently for each occurrence:
R₄ represents alkyl, fluoroalkyl, cycloalkyl, aryl, heteroaryl, aralkyl, heteroaralkyl, or —(CH₂)ₙ—R₈; or ⁺N(R₄)₃ taken together represents pyridinium, imidazolium, benzimidazolium, pyrazolium, benzpyrazolium, indazolium, thiazolium, benzthiazolium, oxazolium, benzoxazolium, isoxazolium, isothiazolium, imdazolidenium, guanidinium, quinuclidinium, triazolium, tetrazolium, quinolinium, isoquinolinium, piperidinium, pyrrolidinium, morpholinium, pyridazinium, pyrazinium, piperazinium, triazinium, azepinium, or diazepinium;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —OC(O)R′, —CO₂R₄, —C(O)N(R″)₂, —C(O)N(R″)N(R″)₂, —N(R′)₂, —OR′, —SR′, —S(O)R″, —S(O)₂R″, —CN, —N(R″)P(O)(R₄)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 1-10 inclusive.

12. The salt of claim 1, wherein the onium cation is represented by formula If: 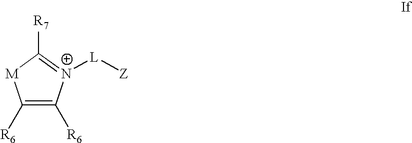
wherein, independently for each occurrence:
M is S or R₅;
R₅ represents alkyl, fluoroalkyl, cycloalkyl, aryl, heteroaryl, aralkyl, heteroaralkyl, or —(CH₂)ₙ—R₈; or R₅ is represented by formula If-a: 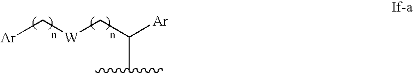
wherein, independently for each occurrence:
Ar represents a substituted or unsubstituted aryl or heteroaryl ring; and
W represents O, NR₇, or S;
R₆ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R₇ represents H, alkyl, fluoroalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —CO₂R₅, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 0-10 inclusive.

13. The salt of claim 12, wherein M is S.

14. The salt of claim 12, wherein M is NR₅ and R₅ represents alkyl.

15. The salt of claim 12, wherein M is NR₅ and R₅ is

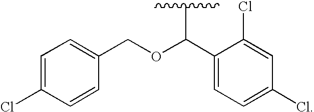

16. The salt of claim 1, wherein the onium cation is represented by formula Ig: 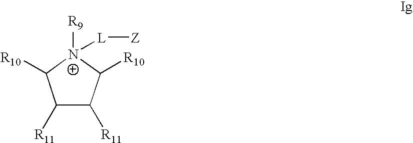
wherein, independently for each occurrence:
R₉ represents alkyl, fluoroalkyl, cycloalkyl, aryl, heteroaryl, aralkyl, heteroaralkyl, or —(CH₂)ₙ—R₈;
R₁₀ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R₁₁ represents H, alkyl, fluoroalkyl, aryl, heteroaryl, aralkyl, heteroaralkyl, or —(CH₂)ₙ—R₈;
L represents (C(R₃)₂)ₙ, (C(R₃)₂)ₙJ(C(R₃)₂)ₘ, or (C(R₃)₂)ₙAr(C(R₃)₂)ₘ;
Z represents H, —CO₂H, —OC(O)R′—, —CO₂R₉, —C(O)N(R″)₂, —C(O)N(R″)N(R″)₂, —N(R′)₂, —OR′, —SR′, —S(O)R″, —S(O)₂R″, —CN, —N(R″)P(O)(R₅)₂, —C(OR′)(R″)₂, alkenyl, or alkynyl;
Ar represents aryl or heteroaryl;
J represents O, S, NR′, cycloalkyl, or heterocyclyl;
R′ represents H, alkyl, fluoroalkyl, aryl, heteroaryl, aralkyl, heteroaralkyl, formyl, acyl, alkyloxycarbonyl, aryloxycarbonyl, alkylaminocarbonyl, arylaminocarbonyl, or —(CH₂)ₙ—R₈;
R″ represents H, alkyl, fluoroalkyl, aryl, heteroaryl, aralkyl, heteroaralkyl, or —(CH₂)ₙ—R₈;
R₃ represents H, F, or alkyl;
R₈ represents cycloalkyl, aryl, or heteroaryl;
m represents an integer from 1-10 inclusive; and
n represents an integer from 1-10 inclusive.

17. The salt of claim 1, wherein the onium cation is represented by formula Ih: 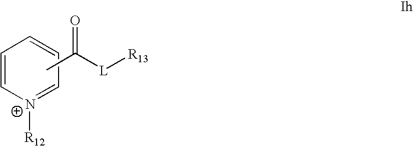
wherein:
L represents O, NR₁₂, or S;
R₁₂ represents H, alkyl, fluoroalkyl, aryl, heteroaryl, aralkyl, heteroaralkyl, or —(CH₂)ₙ—R₈;
R₁₃ represents H, alkyl, fluoroalkyl, aryl, heteroaryl, aralkyl, heteroaralkyl, or —(CH₂)ₙ—R₈; and
R₈ represents cycloalkyl, aryl, or heteroaryl.

18. A heat storage media comprising the salt of any of claims 1 to 17.
